SEQUENCE LISTING (26 sequences)

SEQ ID NO: 1 - protein, 131 aa, Sars-Cov-2
MSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYA

SEQ ID NO: 2 - protein, 277 aa, Sars-Cov-2
MSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFT

SEQ ID NO: 3 - protein, 366 aa, Sars-Cov-2
MSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSR

SEQ ID NO: 4 - protein, 582 aa, Sars-Cov-2
MSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAA

SEQ ID NO: 5 - protein, 683 aa, Sars-Cov-2
ALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 6 - protein, 656 aa, Sars-Cov-2
EERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 7 - protein, 351 aa, Sars-Cov-2
TRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 8 - protein, 119 aa, Sars-Cov-2
LRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTR

SEQ ID NO: 9 - protein, 116 aa, Sars-Cov-2
ALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSR

SEQ ID NO: 10 - protein, 216 aa, Sars-Cov-2
LSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAA

SEQ ID NO: 11 - protein, 818 aa, synthetic construct
MSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 12 - protein, 717 aa, synthetic construct
MSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRTRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 13 - protein, 933 aa, Sars-Cov-2
MSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 14 - protein, 40 aa, synthetic construct
  REGION 1..40: note = synthetic
SHGFPPAVAAQDDGTLPMSCAQESGMDRHPAACASARINV

SEQ ID NO: 15 - protein, 8 aa, Gallus gallus
SIINFEKL

SEQ ID NO: 16 - skipped

SEQ ID NO: 17 - protein, 1162 aa, Kaposi's sarcoma herpesvirus (KSHV)
MAPPGMRLRSGRSTGAPLTRGSCRKRNRSPERCDLGDDLHLQPRRKHVADSIDGRECGPHTLPIPGSPTVFTSGLPAFVSSPTLPVAPIPSPAPATPLPPPALLPPVTTSSSPIPPSHPVSPGTTDTHSPSPALPPTQSPESSQRPPLSSPTGRPDSSTPMRPPPSQQTTPPHSPTTPPPEPPSKSSPDSLAPSTLRSLRKRRLSSPQGPSTLNPICQSPPVSPPRCDFANRSVYPPWATESPIYVGSSSDGDTPPRQPPTSPISIGSSSPSEGSWGDDTAMLVLLAEIAEEASKNEKECSENNQAGEDNGDNEISKESQVDKDDNDNKDDEEEQETDEEDEEDDEEDDEEDDEEDDEEDDEEDDEEDDEEDDEEDDEEDDEEDDEEEDEEEDEEEDEEEEDEEDDDDEDNEDEEDDEEEDKKEDEEDGGDGNKTLSIQSSQQQQEPQQQEPQQQEPQQQEPLQEPQQQEPQQQEPQQQEPLQEPQQQEPQQQEPLQEPQQQEPQQQEPQQQEPQQQEPQQQEPQQQEPQQQEPQQQEPQQQEPQQQEPQQREPQQREPQQREPQQREPQQREPQQREPQQREPQQREPQQREPQQQDEQQQDEQQQDEQQQDEQQQDEQQQDEQQQDEQQQDEQQQDEQQQDEQQQDEQQQDEQQQDEQQQDEQQQDEQQQDEQQQDEQQQDEQQQDEQQQDEQQQDEQEQQDEQEQQDEQEQQDEQQQDEQQQQDEQQQQDEQQQQDEQQQQDEQQQQDEQEQQEEQEQQEEQEQELEEQEQELEDQEQELEEQEQELEEQEQELEEQEQELEEQEQELEEQEQELEEQEQELEEQEQELEEQEQELEEQEVEEQEQEVEEQEQEQEEQELEEVEEQEQEQEEQEEQELEEVEEQEEQELEEVEEQEEQELEEVEEQEQQELEEVEEQEQQGVEQQEQETVEEPIILHGSSSEDEMEVDYPVVSTHEQIASSPPGDNTPDDDPQPGPSREYRYVLRTSPPHRPGVRMRRVPVTHPKKPHPRYQQPPVPYRQIDDCPAKARPQHIFYRRFLGKDGRRDPKCQWKFAVIFWGNDPYGLKKLSQAFQFGGVKAGPVSCLPHPGPDQSPITYCVYVYCQNKDTSKKVQMARLAWEASHPLAGNLQSSIVKFKKPLPLTQPGENQGPGDSPQEMT

SEQ ID NO: 18 - DNA, 3489 bp, synthetic construct
  misc_feature 1..3489: note = synthetic
  source 1..3489: mol_type = other DNA
atggcgcccccgggaatgcgcctgaggtcgggacggagcaccggcgcgcccttaacgagaggaagttgtaggaaacgaaacaggtctccggaaagatgtgaccttggcgatgacctacatctacaaccgcgaaggaagcatgtcgccgactccatcgacggccgggaatgtggaccccacaccttgcctatacctggaagtcccacagtgttcacatccgggctgccagcatttgtgtctagtcctactttaccggtggctcccattccttcacccgctcccgcaacacctttacctccaccggcactcttaccccccgtaaccacgtcttcctccccaatccctccatcccatcctgtgtctccggggaccacggatactcattctccatctcctgcattgccacccacgcagtctccagagtcttctcaaaggccaccgctttcaagtcctacaggaaggccagactcttcaacacctatgcgtccgccaccctcgcagcagactacacctccacactcacccacgactcctccacccgagcctccctccaagtcgtcaccagactctttagctccgtctaccctgcgtagcctgagaaaaagaaggctatcgtccccccaaggtccctctacactaaacccaatatgtcagtcgcccccagtctctccccctagatgtgacttcgccaaccgtagtgtgtaccccccatgggccacagagtccccgatctacgtgggatcatccagcgatggcgatactccgccacgccaaccgcctacatctcccatctccataggatcatcatccccgtctgagggatcctggggtgatgacacagccatgttggtgctccttgcggagattgcagaagaagcatccaagaatgaaaaagaatgttccgaaaataatcaggctggcgaggataatggggacaacgagattagcaaggaaagtcaggttgacaaggatgacaatgacaataaggatgatgaggaggagcaggagacagatgaggaggacgaggaggatgacgaggaggatgacgaggaggatgacgaggaggatgacgaggaggatgacgaggaggatgacgaggaggatgacgaggaggatgacgaggaggatgacgaggaggatgacgaggaggatgacgaggaggaggacgaggaggaggacgaggaggaggacgaggaggaggaggacgaggaggatgacgatgatgaggacaatgaggacgaggaggatgacgaggaggaggacaagaaggaggacgaggaggacgggggcgatggaaacaaaacgttgagcatccaaagttcacaacagcagcaggagccacaacagcaggagccacagcagcaggagccacagcagcaggagcccctgcaggagccacaacagcaggagccacagcagcaggagccacagcagcaggagcccctgcaggagccacaacagcaggagccacagcagcaggagcccctgcaggagccacaacagcaggagccacaacagcaggagccacagcagcaggagccacagcagcaggagccacagcagcaggagccacagcagcaggagccacagcagcaggagccacagcagcaggagccacagcagcaggagccacagcagcaggagccacagcagcgggagccacagcagcgggagccccagcagcgggagccacagcagcgggagccacagcagcgggagccacagcagcgggagccacagcagcgggagccacagcagcgggagccacagcagcgggagccacagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcagcagcaggatgagcaggagcagcaggatgagcaggagcagcaggatgagcaggagcagcaggatgagcagcagcaggatgagcagcagcagcaggatgagcagcagcagcaggatgagcagcagcagcaggatgagcagcagcagcaggatgagcagcagcagcaggatgaacaggagcagcaggaggagcaggagcagcaggaggagcaggagcaggagttagaggagcaggagcaggagttagaggatcaggagcaggagttagaggagcaggagcaggagttagaggagcaggagcaggagttagaggagcaggagcaggagttagaggagcaggagcaggagttagaggagcaggagcaggagttagaggagcaggagcaggagttagaggagcaggagcaggagttagaggagcaggagcaggagttagaggagcaggaggtggaagagcaagagcaggaggtggaagagcaagagcaggagcaggaagagcaggaattagaggaggtggaggagcaagagcaggagcaggaggagcaggaggagcaggagttagaggaggtggaagagcaggaagagcaggagttagaggaggtggaagagcaggaagagcaggagttagaggaggtggaagagcaggagcagcaggagttagaggaggtggaagagcaggagcagcagggggtggaacagcaggagcaggagacggtggaagagcccataatcttgcacgggtcgtcatccgaggacgaaatggaagtggattaccctgttgttagcacacatgaacaaattgccagtagcccaccaggagataatacaccagacgatgacccacaacctggcccatctcgcgaataccgctatgtactcagaacatcaccaccccacagacctggagttcgtatgaggcgcgttccagttacccacccaaaaaagccacatccaagataccaacaaccaccggtcccttacagacagatagatgattgtcctgcgaaagctaggccacaacacatcttttatagacgctttttgggaaaggatggaagacgagatccaaagtgtcaatggaagtttgcagtgattttttggggcaatgacccatacggacttaaaaaattatctcaggccttccagtttggaggagtaaaggcaggccccgtgtcctgcttgccccaccctggaccagaccagtcgcccataacttattgtgtatatgtgtattgtcagaacaaagacacaagtaagaaagtacaaatggcccgcctagcctgggaagctagtcaccccctggcaggaaacctacaatcttccatagttaagtttaaaaagcccctgccattaacccagccaggggaaaaccaaggtcctggggactctccacaggaaatgacataa

SEQ ID NO: 19 - DNA, 36 bp, synthetic construct
  misc_feature 1..36: note = synthetic
  source 1..36: mol_type = other DNA
ggatccgccaccatggtgagcaagggcgaggagctg

SEQ ID NO: 20 - DNA, 30 bp, synthetic construct
  misc_feature 1..30: note = synthetic
  source 1..30: mol_type = other DNA
gaattccacattgatcctagcagaagcaca

SEQ ID NO: 21 - DNA, 48 bp, synthetic construct
  misc_feature 1..48: note = synthetic
  source 1..48: mol_type = other DNA
aagcttagcataattaatttcgaaaagctctaagcggccgcgctcgag

SEQ ID NO: 22 - DNA, 48 bp, synthetic construct
  misc_feature 1..48: note = synthetic
  source 1..48: mol_type = other DNA
ctcgagcgcggccgcttagagcttttcgaaattaattatgctaagctt

SEQ ID NO: 23 - protein, 20 aa, synthetic construct
  REGION 1..20: note = synthetic
GGGGSGGGGSGGGGSGGGGS

SEQ ID NO: 24 - protein, 15 aa, synthetic construct
  REGION 1..15: note = synthetic
GGGGSGGGGSGGGGS

SEQ ID NO: 25 - protein, 5 aa, synthetic construct
  REGION 1..5: note = synthetic
GGGGS

SEQ ID NO: 26 - skipped

(19) United States
(12) Patent Application Publication
(10) Pub. No.: US 2025/0352641 A1
(43) Pub. Date: Nov. 20, 2025

(54) METHODS TO INCREASE IMMUNOGENICITY OF RDRP

(71) Applicant: UNIVERSITY OF PITTSBURGH- OF THE COMMONWEALTH SYSTEM OF HIGHER EDUCATION, Pittsburgh, PA (US)

(72) Inventors: Patrick S. Moore, Pittsburgh, PA (US); Yuan CHANG, Pittsburgh, PA (US); Li WAN, Pittsburgh, PA (US)

(21) Appl. No.: 18/863,216

(22) PCT Filed: May 4, 2023
(86) PCT No.: PCT/US2023/066604

§ 371 Date: Nov. 5, 2024

Related U.S. Application Data
(60) Provisional application No. 63/339,186, filed on May 6, 2022.

(51) Int. Cl. A61K 39/215; A61K 39/00; C12N 9/12

(52) U.S. Cl. CPC A61K 39/215; C12N 9/127; C12Y 207/07048; A61K 2039/53; C07K 2319/50; C12N 2770/20022; C12N 2770/20034

(57) ABSTRACT

Disclosed are recombinant vectors comprising one or more polynucleotides encoding at least one modified ribonucleic acid (RNA) dependent RNA polymerase (RdRp), as well as pharmaceutical compositions and methods of their use.

### CROSS REFERENCE TO RELATED APPLICATIONS

This patent application is the U.S. National Phase of co-pending International Patent Application No. PCT/US2023/066604, filed May 4, 2023, which claims the benefit of U.S. Provisional Patent Application No. 63/339,186, filed May 6, 2022, both of which are hereby incorporated by reference in their entireties herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 36,260 Byte XML file named “771649_ST26 replacement.XML,” created Aug. 4, 2025.

### BACKGROUND OF THE INVENTION

Vaccines have proven to be effective mitigators of viral damage. Although there have been many recent advances in vaccine technology, viral infections continue to plague society and cause significant morbidity and mortality. There is also a lack of effective treatments available for many viral infections. Accordingly, there is a need for improved vaccines.

### BRIEF SUMMARY OF THE INVENTION

Aspects of the invention provide recombinant vectors comprising one or more polynucleotides encoding at least one modified viral ribonucleic acid (RNA) dependent RNA polymerase (RdRp), wherein the modified RdRp comprises: (a) at least one fragment of RdRp; (b) RdRp and a regulatory protein that enhances proteasomal degradation, wherein the RdRp is conjugated or fused to the regulatory protein directly or indirectly via a linker; (c) RdRp and a peptide sequence rich in proline (P), glutamic acid (E), serine (S), and threonine (T) (PEST sequence) that enhances proteasomal degradation; or (d) a combination of two or more of (a)-(c).

An aspect of the invention provides pharmaceutical compositions comprising the vectors described herein and a pharmaceutically acceptable carrier.

An aspect of the invention provides methods of destabilizing SARS-CoV-2 RdRp in a cell, the methods comprising contacting a cell infected with SARS-CoV-2 virus vectors described herein.

An aspect of the invention provides methods of increasing immune response to SARS-CoV-2 virus in a subject, the methods comprising administering the vectors or pharmaceutical compositions described herein to the subject.

An aspect of the invention provides methods of increasing cytotoxic T lymphocyte (CTL) response to SARS-CoV-2 virus in a subject, the methods comprising administering the vectors or pharmaceutical compositions described herein to the subject.

An aspect of the invention provides methods of increasing Major Histocompatibility complex (MHC) class I immune response to SARS-CoV-2 virus in a subject, the methods comprising administering the vectors or pharmaceutical compositions described herein to the subject.

### BRIEF DESCRIPTION OF THE SEVERAL VIEWS OF THE DRAWING(S)

FIG. 1A includes schematic showing a 5,059 base pair circular plasmid named “pCMV-Tag 2B-EGFP-SIINFEKL.” pCMV-Tag 2B-EGFP-SIINFEKL contains EGFP to test expression via fluorescence and SIINFEKL (OVA257-264 peptide, SEQ ID NO: 15) to test MHC class I peptide presentation. pCMV-Tag 2B-EGFP-SIINFEKL also contains a human cytomegalovirus (CMV) enhancer, a CMV promoter, a T3 promoter, a multiple cloning site (MCS), ATG start codon, a FLAG-tag, stop codons, a T7 promoter, a SV40 poly(A) signal, an f1 origin of replication (f1 ori), an AmpR promoter, a NeoR/KanR, a HSV TK poly(A) signal, and origin of replication (ori) sequences. pCMV-Tag 2B-EGFP-SIINFEKL was transfected into 293H2Kb cells for protein expression. The top of FIG. 1A shows a schematic of EGFP fused to SIINFEKL (SEQ ID NO: 15).

FIG. 1B includes a schematic showing a 7,821 base pair circular plasmid named “pCMVTag2B-RdRp-EGFP-SIINFEKL.” pCMVTag2B-RdRp-EGFP-SIINFEKL contains EGFP, SIINKEKL (SEQ ID NO: 15), and SarS-CoV-2 RdRp (non-structural protein subunit 12 (nsp12), codon-optimized). pCMVTag2B-RdRp-EGFP-SIINFEKL also contains a human CMV enhancer, a CMV promoter, a T3 promoter, a Kozak consensus, stop codons, a T7 promoter, a SV40 poly(A) signal, an f1 ori, an AmpR promoter, an SV40 promoter, a NeoR/KanR, a HSV TK poly(A) signal, and ori sequences. The SarS-CoV-2 RdRp sequence contains Fbw7 degron, APCC1, anaphase-promoting complex or cyclosome destruction box (APC/C 2 (D Box)), APCC3 (D Box), and APCC4 (ABBAyCDC20). The top of FIG. 1B shows a schematic of RdRp fused to EGFP and EGFP fused to SIINFEKL (SEQ ID NO: 15).

FIG. 2 is an exemplary gel following a Western blot analysis that screened for protein expressions. pCMVTag2B-EGFP was a positive control (vector plus florescent protein), pCMVTag2B-EGFP-SIINFEKL was the positive control for MHC class I presentation, pCMVTag2B-RdRp-EGFP-SIINFEKL was the target gene, pCMVTag2B-EGFP-LANA-SIINFEKL was the negative control for MHC class I presentation, pCMV-Tag2B was the negative control, i.e., the empty vector, pCMVTag2B-d1EGFP-SIINFEKL and pCMVTag2B-RdRp-d1EGFP-SIINFEKL contained a PEST sequence. Beta-Tubulin (Tubulin) was used as loading control. LANA refers to Kaposi's sarcoma-associated herpesvirus (KSHV) latency-associated nuclear antigen 1 (LANAI, SEQ ID NO: 17).

FIG. 3 is a set of images showing EGFP expression of vectors of aspects of the present invention on a fluorescence microscope.

FIGS. 4A-4C are sets of flow cytometry plots showing the EGFP levels versus SIINFEKL (SEQ ID NO: 15) presentation level of exemplary samples. In FIG. 4A, the percentages at the top of each plot (i.e., 2%, 6%, 10%, 20%, 40%, and 100%) refer to the gradient of vector concentration EGFP-SIINFEKL (SEQ ID NO: 15), whereas 100% indicates 2 μg of plasmid transfected to 40,000 cells in a 6-well plate by 2 μl of lipofectamine 2000 (Invitrogen). In FIG. 4B, the percentages at the top of each plot (i.e., 6% and 15%) refers to the gradient of vector concentration EGFP-LANAI-SIINFEKL (SEQ ID NO: 15). In FIG. 4C, the percentages at the top of each plot (i.e., 100%) refers to the gradient of vector concentration RdRp-EGFP-SIINFEKL (SEQ ID NO: 15). This analysis plot was run in duplicate.

FIG. 5A is a set of schematics showing aspects of the present invention. From top to bottom, the schematics show: (1) ubiquitin fused to EGFP fused to SIINFEKL (SEQ ID NO: 15); (2) ubiquitin fused to RdRp fused to EGFP fused to SIINFEKL (SEQ ID NO: 15); (3) ubiquitin fused to an A-R linker fused to EGFP fused to SIINFEKL (SEQ ID NO: 15); and (4) ubiquitin fused to an A-R linker fused to RdRp fused to EGFP fused to SIINFEKL (SEQ ID NO: 15).

FIG. 5B is schematic showing an aspect of the present invention. Specifically, it shows a fusion protein with RdRp fused to SIINFEKL (SEQ ID NO: 15) fused to EGFP fused to a PEST sequence.

FIG. 5C is a schematic showing an aspect of the present invention. Specifically, it shows two fusion proteins that were used together (SEQ ID NOs: 10 and 12) fused to EGFP fused to SIINFEKL (SEQ ID NO: 15), and the second (bottom) fusion protein with amino acids 366-581 of the full length RdRp (SEQ ID NO: 13) fused to EGFP fused to SIINFEKL (SEQ ID NO: 15).

FIG. 6A is a set of schematics showing aspects of the present invention by adding a ubiquitin to RdRp N-terminus.

FIG. 6B is a set of flow cytometry plots showing MHC class I presentation of SIINFEKL (SEQ ID NO: 15) peptide in ubiquitin containing RdRp fusion proteins of FIG. 6A. SIINFEKL (SEQ ID NO: 15) presentation increased from 1.13% to 2.83% by ubiquitin tag.

FIG. 7A is a set of schematics showing aspects of the present invention by adding a ubiquitin as well as an Alanine-Arginine linker.

FIG. 7B is a set of flow cytometry plots showing MHC class I presentation of SIINFEKL (SEQ ID NO: 15) peptide in ubiquitin containing RdRp fusion proteins of FIG. 7A. The ubiquitin-A-R increased SIINFEKL (SEQ ID NO: 15) presentation from 1.13% to 4.88%.

FIG. 8A is a set of schematics showing aspects of the present invention by addition of a PEST sequence.

FIG. 8B is a set of flow cytometry plots showing the MHC class I presentation of SIINFEKL (SEQ ID NO: 15) peptide of the PEST containing RdRp fusion proteins of FIG. 8A. The PEST sequence increased SIINFEKL (SEQ ID NO: 15) presentation from 5.11% to 7.97%.

FIG. 9 is a schematic showing the layout of RdRp fragment constructs of aspects of the present invention that were inserted in the N-terminal of EGFP-SIINFEKL (SEQ ID NO: 15).

FIG. 10 is an exemplary gel following a Western blot analysis that screened for expression of RdRp fragment constructs of aspects of the present invention.

FIG. 11 is a set of flow cytometry plots showing EGFP level and SIINFEKL (SEQ ID NO: 15) presentation of fragmented RdRp-EGFP-SIINFEKL constructs.

FIG. 12 is a set of flow cytometry plots showing EGFP level and SIINFEKL (SEQ ID NO: 15) presentation of fragmented RdRp-EGFP-SIINFEKL constructs. Compared with FIG. 11, most fragmented RdRp constructs showed increased SIINFEKL (SEQ ID NO: 15) presentation. SIINFEKL (SEQ ID NO: 15) presentation was increased from 11% for RdRp (FL) (SEQ ID NO: 13) up to 24.6% for RdRp.aa.1-130 construct (SEQ ID NO: 1).

FIG. 13 is a set of flow cytometry plots showing EGFP level and SIINFEKL (SEQ ID NO: 15) presentation of fragmented RdRp-EGFP-SIINFEKL constructs. It further shows that all small fragments (<200 bp) could dramatically increase SIINFEKL (SEQ ID NO: 15) presentation, up to 30.1% for RdRp.aa.366-581 construct (SEQ ID NO: 10).

FIG. 14 is a graph comparing the EGFP level and SIINFEKL (SEQ ID NO: 15) presentation of fragmented RdRp-EGFP-SIINFEKL constructs and full length RdRp-EGFP-SIINFEKL. The majority of fragments showed increased SIINFEKL (SEQ ID NO: 15) presentation.

FIG. 15A is a flow cytometry plot showing RdRp fragments used in combination for SIINFEKL (SEQ ID NO: 15) presentation. All fragment combinations showed increased SIINFEKL (SEQ ID NO: 15) presentation, even when the protein expression level is comparable.

FIG. 15B is a flow cytometry plot showing RdRp fragments used in combination for SIINFEKL (SEQ ID NO: 15) presentation. All fragment combinations showed increased SIINFEKL (SEQ ID NO: 15) presentation, even when the protein expression level is comparable.

FIG. 15C is a flow cytometry plot showing RdRp fragments used in combination for SIINFEKL (SEQ ID NO: 15) presentation. All fragment combinations showed increased SIINFEKL (SEQ ID NO: 15) presentation, even when the protein expression level is comparable.

FIG. 16A is a flow cytometry plot showing EGFP-SIINFEKL and EGFP-LANA1 as a control for RdRp-EGFP-SIINFEKL.

FIGS. 16B is a flow cytometry plot showing EGFP-SIINFEKL and EGFP-LANA1 as a control for RdRp-EGFP-SIINFEKL.

FIG. 17A is a flow cytometry plot showing Hsp70-EGFP-SIINFEKL as a control for RdRp-EGFP-SIINFEKL.

FIG. 17B is a flow cytometry plot showing Hsp70-EGFP-SIINFEKL as a control for RdRp-EGFP-SIINFEKL.

FIG. 18 is a graph showing GFP versus SIINFEKL (SEQ ID NO: 15) level and fitting lines for the shown constructs. The higher slope of the fitting line indicates higher SIINFEKL (SEQ ID NO: 15) presentation at the same GFP level (protein expression level).

FIG. 19A is a flow cytometry plot showing the results of analysis of constructs of aspects of the present invention at gradient (x3) concentrations.

FIG. 19B is a flow cytometry plot showing the results of analysis of constructs of aspects of the present invention at gradient (x3) concentrations.

FIG. 19C is a flow cytometry plot showing the results of analysis of constructs of aspects of the present invention at gradient (x3) concentrations.

FIG. 19D is a flow cytometry plot showing the results of analysis of constructs of aspects of the present invention at gradient (x3) concentrations.

FIG. 20A is a flow cytometry plot showing the results of analysis of constructs of aspects of the present invention at gradient (x3) concentrations.

FIG. 20B is a flow cytometry plot showing the results of analysis of constructs of aspects of the present invention at gradient (x3) concentrations.

FIG. 20C is a flow cytometry plot showing the results of analysis of constructs of aspects of the present invention at gradient (x3) concentrations.

FIG. 20D is a flow cytometry plot showing the results of analysis of constructs of aspects of the present invention at gradient (x3) concentrations.

FIG. 20E is a flow cytometry plot showing the results of analysis of constructs of aspects of the present invention at gradient (x3) concentrations.

FIG. 20F is a flow cytometry plot showing the results of analysis of constructs of aspects of the present invention at gradient (x3) concentrations.

FIG. 21 is a graph showing GFP versus SIINFEKL (SEQ ID NO: 15) level and fitting lines for the shown constructs (1st). The higher slope of the fitting line indicates higher SIINFEKL (SEQ ID NO: 15) presentation at the same GFP level (protein expression level). This graph is similar to FIG. 18 but at a lower overall dose.

FIG. 22A is a flow cytometry plot showing the results of analysis of constructs of aspects of the present invention at gradient (x3) concentrations.

FIG. 22B is a flow cytometry plot showing the results of analysis of constructs of aspects of the present invention at gradient (x3) concentrations.

FIG. 22C is a flow cytometry plot showing the results of analysis of constructs of aspects of the present invention at gradient (x3) concentrations.

FIG. 22D is a flow cytometry plot showing the results of analysis of constructs of aspects of the present invention at gradient (x3) concentrations.

FIG. 23A is a flow cytometry plot showing the results of analysis of constructs of aspects of the present invention at gradient (x3) concentrations.

FIG. 23B is a flow cytometry plot showing the results of analysis of constructs of aspects of the present invention at gradient (x3) concentrations.

FIG. 23C is a flow cytometry plot showing the results of analysis of constructs of aspects of the present invention at gradient (x3) concentrations.

FIG. 24A is a flow cytometry plot showing the results of analysis of constructs of aspects of the present invention at gradient (x3) concentrations.

FIG. 24B is a flow cytometry plot showing the results of analysis of constructs of aspects of the present invention at gradient (x3) concentrations.

FIG. 25 is a graph showing GFP versus SIINFEKL (SEQ ID NO: 15) level and fitting lines for the shown constructs (2nd).

FIG. 26A is a flow cytometry plot showing GFP versus SIINFEKL (SEQ ID NO: 15) level expression of constructs of aspects of the present invention transfected into B16 melanoma cells (human).

FIG. 26B is a flow cytometry plot showing GFP versus SIINFEKL (SEQ ID NO: 15) level expression of constructs of aspects of the present invention transfected into B16 melanoma cells (human).

FIG. 26C is a flow cytometry plot showing GFP versus SIINFEKL (SEQ ID NO: 15) level expression of constructs of aspects of the present invention transfected into B16 melanoma cells (human).

FIG. 26D is a flow cytometry plot showing GFP versus SIINFEKL (SEQ ID NO: 15) level expression of constructs of aspects of the present invention transfected into B16 melanoma cells (human).

FIG. 26E is a flow cytometry plot showing GFP versus SIINFEKL (SEQ ID NO: 15) level expression of constructs of aspects of the present invention transfected into B16 melanoma cells (human).

### DETAILED DESCRIPTION OF THE INVENTION

The majority of viruses infecting humans and other animals have RNA genomes. These genomes may be double-stranded (ds) or single-stranded (ss). The RNA-dependent RNA polymerase (RdRp) of all known single-stranded RNA viruses is located within the viral particle and is responsible for the transcription and replication of the viral genome. Presenting destabilized RdRp in a recombinant vector to a viral host has been found to increase the MHC class I presentation which indicates promising use of destabilized RdRp to increase the protection of the host from the virus. The RdRp can be destabilized by modifying the RdRp in several ways.

Specifically, an aspect of the invention provides recombinant vectors comprising one or more polynucleotides encoding at least one modified RdRp, wherein the modified RdRp comprises: (a) at least one fragment of RdRp; (b) RdRp and a regulatory protein that enhances proteasomal degradation, wherein the RdRp is conjugated or fused to the regulatory protein directly or indirectly via a linker; (c) RdRp and a peptide sequence rich in proline (P), glutamic acid (E), serine (S), and threonine (T) (PEST sequence) that enhances proteasomal degradation; or (d) a combination of two or more of (a)-(c) (i.e., two or three of (a)-(c)).

The RdRp can be from any single-stranded RNA virus. A “single-stranded RNA virus” comprises a single stranded RNA genome. Corona viruses are single-stranded RNA viruses.

In some aspects, the RdRp is a corona virus RdRp. In at least one aspect, the RdRp is a SARS-CoV-2 RdRp. In an aspect, the corona virus is Middle East Respiratory Syndrome (MERS)-CoV. In an aspect, the corona virus is severe acute respiratory syndrome (SARS)-CoV. In an aspect, the corona virus is an alpha, beta, gamma, delta, or omicron type of corona virus. In an aspect, the corona virus is 229E (alpha coronavirus), NL63 (alpha coronavirus), OC43 (beta coronavirus), or HKU1 (beta coronavirus). In an aspect, the corona virus is SARS-CoV-1 or SARS-CoV-2. In an aspect, the corona virus is SARS-CoV-2. In an aspect, the corona virus is SARS-CoV-2 variant alpha (B.1.1.7 and Q lineages), beta (B.1.351 and descendent lineages), gamma (P.1 and descendent lineages), epsilon (B.1.427 and B.1.429), eta (B.1.525), iota (B.1.526), kappa (B.1.617.1), 1.617.3, mu (B.1.621, B.1.621.1), zeta (P.2), delta (B.1.617.2 and AY lineages), or omicron (B.1.1.529 and BA lineages). In an aspect, the corona virus is historically a non-human animal corona virus that evolves, mutates, or is modified to cause infection in a human.

The RdRp used can be a full-length or a fragment of a full-length RdRp. In an aspect, the at least one fragment of RdRp comprises at least about 100 amino acids (e.g., comprises at least about 105 amino acids, at least about 110 amino acids, at least about 115 amino acids, at least about 120 amino acids, at least about 125 amino acids, at least about 130 amino acids, at least about 135 amino acids, at least about 140 amino acids, at least about 145 amino acids). In another aspect, the at least one fragment of RdRp comprises no more than about 950 amino acids (e.g., comprises no more than about 960 amino acids, no more than about 970 amino acids, no more than about 980 amino acids, no more than about 990 amino acids, no more than about 1,000 amino acids, or no more than about 1,100 amino acids). In an aspect, the at least one fragment of RdRp comprises from about 100 amino acids to about 950 amino acids, from about 125 amino acids to about 900 amino acids, from about 150amino acids to about 850 amino acids, from about 175 amino acids to about 800 amino acids, or from about 200 amino acids to about 750 amino acids.

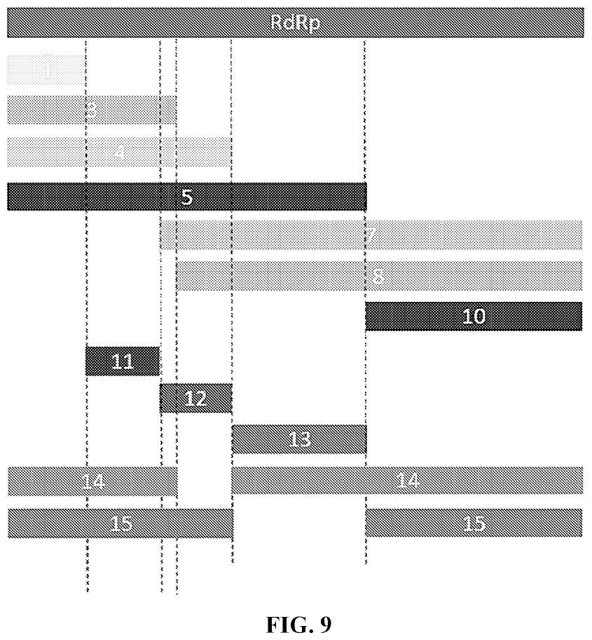

In an additional aspect, the at least one fragment of RdRp comprises at least one of SEQ ID NOs: 1-12. In a further aspect, the vector of an aspect of the invention comprises: (a) SEQ ID NOs: 1, 8, and 9; (b) SEQ ID NOs: 2 and 6; (c) SEQ ID NOs: 4 and 7; (d) SEQ ID NOs: 10 and 12; (e) SEQ ID NOs: 1, 7, 8, 9, and 10; or (f) a combination of two or more (a)-(e) (i.e., two, three, four, or five of (a)-(e)). FIG. 9 shows how exemplary fragments of RdRp present in the full length RdRp of Sars-Cov-2. Table 1 below provides the RdRp fragment number, SEQ ID NO, size, and corresponding amino acids of the full length RdRp of Sars-Cov-2.

TABLE 1

| SEQ ID | RdRp Fragment | | |
|---|---|---|---|
| NO | Number | Portion of RdRp | Size (kbp) |
| 13 | WT | aa 1-932 (full length) | 2.8 |
| 1 | 1 | RdRp aa 1-130 | 0.4 |
| 2 | 3 | RdRp aa 1-276 | 0.8 |
| 3 | 4 | RdRp aa 1-365 | 1.1 |
| 4 | 5 | RdRp aa 1-581 | 1.7 |
| 5 | 7 | RdRp aa 250-932 | 2.1 |
| 6 | 8 | RdRp aa 277-932 | 2 |
| 7 | 10 | RdRp aa 582-932 | 1.1 |
| 8 | 11 | RdRp aa 131-249 | 0.4 |
| 9 | 12 | RdRp aa 250-365 | 0.3 |
| 10 | 13 | RdRp aa 366-581 | 0.6 |
| 11 | 14 | Full length RdRp | 2.5 |
| | | with aa 251-365 deleted | |
| 12 | 15 | Full length RdRp | 2.2 |
| | | with aa 366-581 deleted | |

In an aspect, more than one fragment of a RdRp is in a single vector. In a further aspect, two fragments of a RdRp are used, wherein the two portions together provide the amino acid sequences of the full length RdRp. For example, RdRp fragment 14 of Sars-Cov-2 is full length RdRp with amino acids 251-365 deleted (SEQ ID NO: 11) and fragment 12 of Sars-Cov-2 is 250-365 amino acids of Sars-Cov-2 full length RdRp (SEQ ID NO: 9). Together, SEQ ID NOs: 9 and 11 provide the amino acid sequences of the full length RdRp.

In a further aspect, the vector comprises a regulatory protein. In an aspect, the regulatory protein is ubiquitin or a ubiquitin-like regulatory protein (e.g., ISG15, NEDD8, and SUMO). In an aspect, the regulatory protein is ubiquitin. In an aspect, the regulatory protein modifies the function of RdRp when the regulatory protein is conjugated or fused to RdRp via a linker by enhancing proteasomal degradation of RdRp.

In an aspect, the regulatory protein is conjugated to the N-terminus of RdRp. In an aspect, the regulatory protein is directly conjugated to the N-terminus of RdRp. In an aspect, the regulatory protein is indirectly conjugated to the N-terminus of RdRp via a linker. In an aspect, the regulatory protein is fused to the N-terminus of RdRp. In an aspect, the regulatory protein is directly fused to the N-terminus of RdRp. In an aspect, the regulatory protein is indirectly fused to the N-terminus of RdRp via a linker.

In an aspect, the regulatory protein is conjugated to the C-terminus of RdRp. In an aspect, the regulatory protein is directly conjugated to the C-terminus of RdRp. In an aspect, the regulatory protein is indirectly conjugated to the C-terminus of RdRp via a linker. In an aspect, the regulatory protein is fused to the C-terminus of RdRp. In an aspect, the regulatory protein is directly fused to the C-terminus of RdRp. In an aspect, the regulatory protein is indirectly fused to the C-terminus of RdRp via a linker.

In an aspect, the linker that is conjugated or fused to RdRp is an Alanine-Arginine linker. An “Alanine-Arginine linker,” “A-R linker,” and “A-R,” refer to a linker that comprises at least one alanine and at least one arginine. The linker can be two or more amino acids in length and a suitable linker will be long enough to prevent misfolding of RdRp protein and the regulatory protein. In an aspect, the Alanine-Arginine linker comprises from 2 to 8 amino acids, from 2 to 6 amino acids, or from 2 to 4 amino acids.

In a further aspect, the vectors comprise a peptide sequence rich in proline (P), glutamic acid (E), serine (S), and threonine (T) (PEST sequence) that enhances proteasomal degradation.

In an aspect, the PEST sequence is a mutated version of residues 422-461 of mouse ornithine decarboxylase (residue numbers refer to the full-length sequence of the mouse ornithine decarboxylase). See, e.g., Li, et al., J. of Biological Chem., 273(52): 34970-34975 (1998); Kitsera, et al., Biotechniques, 43(2): 222-227 (2007); and Kwun, et al., Virology, 412(2): 357-365 (2011).

In an aspect, the PEST sequence comprises at least 10 (e.g., at least 11, at least 12, at least 13, at least 14, at least 15, at least 16, at least 17, at least 18, at least 19, at least 20, at least 25, or at least 30) amino acids. In a further aspect, at least 50% (e.g., at least 55%, at least 60%, at least 65%, at least 70%, at least 75%, at least 80%, at least 85%, at least 90%, or at least 95%) of the total amino acids in the PEST sequence comprise P, E, S, or T.

In an aspect, the PEST sequence has at least about 90% sequence identity to SEQ ID NO: 14 (e.g., at least about 91%, at least about 92%, at least about 93%, at least about 94%, at least about 95%, at least about 96%, at least about 97%, at least about 98%, or at least about 99% sequence identity to SEQ ID NO: 14). In an aspect, the PEST sequence consists essentially of or consists of 90%, 91%, 92%, 93%, 94%, 95%, 96%, 97%, 98%, 99%, or 100% sequence identity to SEQ ID NO: 14.

In an aspect, the PEST sequence is conjugated to the N-terminus of RdRp. In an aspect, the PEST sequence is directly conjugated to the N-terminus of RdRp. In an aspect, the PEST sequence is indirectly conjugated to the N-terminus of RdRp via a linker. In an aspect, the PEST sequence is fused to the N-terminus of RdRp. In an aspect, the PEST sequence is directly fused to the N-terminus of RdRp. In an aspect, the PEST sequence is indirectly fused to the N-terminus of RdRp via a linker.

In an aspect, the PEST sequence is conjugated to the C-terminus of RdRp. In an aspect, the PEST sequence is directly conjugated to the C-terminus of RdRp. In an aspect, the PEST sequence is indirectly conjugated to the C-terminus of RdRp via a linker. In an aspect, the PEST sequence is fused to the C-terminus of RdRp. In an aspect, the PEST sequence is directly fused to the C-terminus of RdRp. In an aspect, the PEST sequence is indirectly fused to the C-terminus of RdRp via a linker.

In an aspect, there is a “Glycine-Serine” linker between RdRp and EGFP in a construct. In an aspect, the Glycine-Serine linker may contain various quantities of amino acid residues, e.g., GGGGSGGGGSGGGGSGGGGS (SEQ ID NO: 23) or GGGGSGGGGSGGGGS (SEQ ID NO: 24). In an aspect, the Glycine-Serine linker may have GGGGS (SEQ ID NO: 25) or GGGGA repeats (SEQ ID NO: 26). In some aspects the Glycine-Serine linker comprises 1, 2, 3, 4, 5, 6, 7, 8, 9, or 10 GGGGS (SEQ ID NO: 25) or GGGGA repeats (SEQ ID NO: 26).

As aspect of the invention also relates to pharmaceutical compositions comprising the vectors of the present invention and a pharmaceutically acceptable carrier. The pharmaceutically acceptable carrier (or excipient) is preferably one that is chemically inert to the vector and one that has no detrimental side effects or toxicity under the conditions of use. Such pharmaceutically acceptable carriers include, but are not limited to, water, saline, Cremophor EL (Sigma Chemical Co., St. Louis, MO), propylene glycol, polyethylene glycol, alcohol, and combinations thereof. The choice of carrier will be determined in part by the particular vector, as well as by the particular method used to administer the composition. Accordingly, there is a wide variety of suitable formulations of the composition. Methods for preparing administrable compositions are known or apparent to those skilled in the art and are described in more detail in, for example, Remington: The Science and Practice of Pharmacy, 22^{nd} Ed., Pharmaceutical Press (2012). In an aspect, the pharmaceutical composition is formulated for subcutaneous injection.

An aspect of the invention also relates to methods of destabilizing SARS-CoV-2 RdRp in a cell, the methods comprising contacting a cell with the vectors of the present invention.

An aspect of the invention also relates to methods of increasing immune response to SARS-CoV-2 virus in a subject, the methods comprising administering the vectors or the pharmaceutical compositions of the present invention to the subject. The increase in immune response (e.g., CTL response, etc.) refers to a comparison between subjects that have and have not been administered the vectors or the pharmaceutical compositions of the present invention.

An aspect of the invention also relates to methods of increasing CTL response to SARS-CoV-2 virus in a subject, the methods comprising administering the vectors or the pharmaceutical compositions of the present invention to the subject. The immune system of the subject responds to the vectors by stimulating an immune response comprising the production of CTLs. The increase in CTL response refers to a comparison between subjects that have and have not been administered the vectors or the pharmaceutical compositions of the present invention.

An aspect of the invention also relates to methods of increasing MHC class I immune response to SARS-CoV-2 virus in a subject, the methods comprising administering the vectors or the pharmaceutical compositions of the present invention the subject. The increase in MHC class I immune response refers to a comparison between subjects that have and have not been administered the vectors or the pharmaceutical compositions of the present invention.

T cells are antigen specific immune cells that function in response to specific antigen signals. T cells do not respond to antigens in a free or soluble form. For a T cell to respond to an antigen, it requires the antigen to be bound to a MHC.

MHC proteins provide the means by which T cells differentiate native or “self” cells from foreign cells. There are two types of MHC, class I MHC and class II MHC. Cytolytic T cells (CD8+) predominately interact with class I MHC proteins. Both MHC complexes are transmembrane proteins with a majority of their structure on the external surface of the cell. Additionally, both classes of MHC have a peptide binding cleft on their external portions. It is in this cleft that small fragments of proteins, native or foreign, are bound and presented to the extracellular environment. T cells specific for the peptide bound to a recognizable MHC complex bind to these MHC-peptide complexes and proceed to the next stages of the immune response.

The vector, or pharmaceutical composition thereof, enhances the immunogenicity (particularly the CTL immunogenicity) for a broad spectrum vaccine, e.g., a SARS-CoV-2 vaccine.

The vector, or pharmaceutical composition thereof, can be administered to a subject by any suitable route including, but not limited to, parental (subcutaneous, intramuscular, intradermal, intraperitoneal, intrathecal, intravenous, and intratumoral), topical, oral, or local administration. In an aspect, the vector is administered subcutaneously.

In an aspect, the vector, or pharmaceutical composition thereof, is administered one time to the subject. In another aspect, the vector is administered more than once to the subject (i.e., multiple times, two, three, four, or more times to the subject).

In another aspect, more than one vector is administered to the subject. For example, one vector may comprise polynucleotides encoding RdRp fragment 5 (SEQ ID NO: 4) and a second vector may comprise polynucleotides encoding RdRp fragment 10 (SEQ ID NO: 7).

In an aspect of the invention, the subject is a mammal, such as a non-human mammal including a mouse, rat, guinea pig, hamster, rabbit, cat, dog, pig, cow, horse, a non-human primate, or a human. In an aspect, the subject is a human.

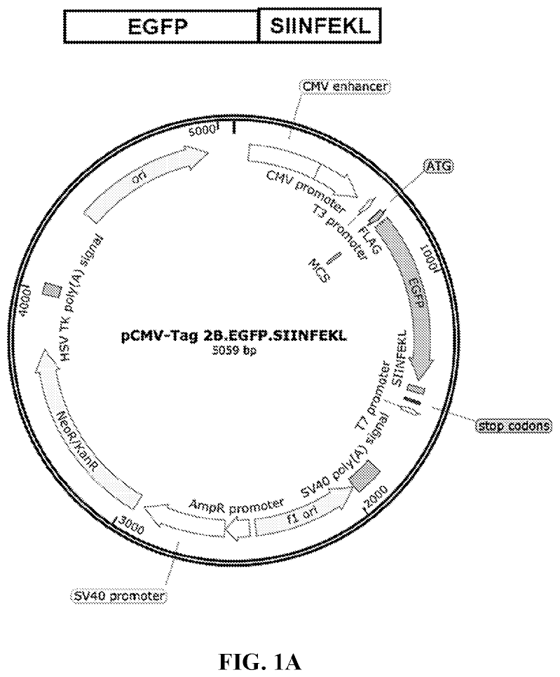

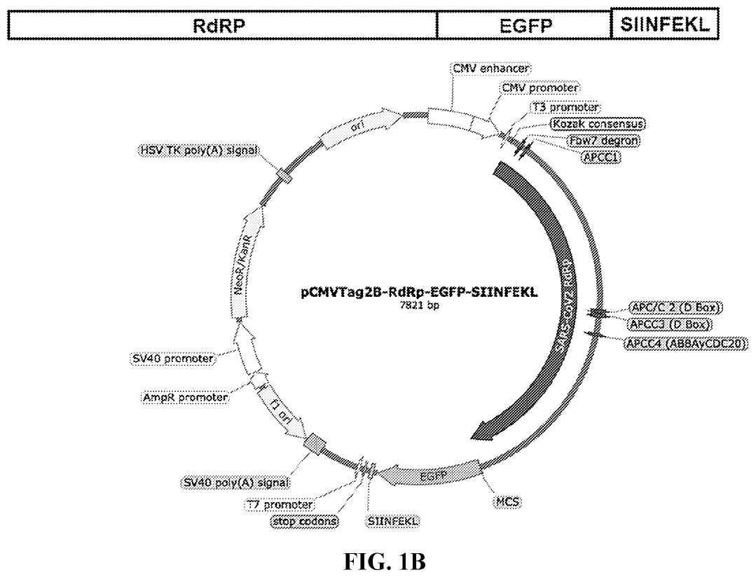

An aspect of the invention provides circular plasmids. Exemplary plasmids are provided in FIGS. 1A and 1B. pCMV-Tag 2B.EGFP.SIINFEKL is a 5,059 base pair circular plasmid and contains EGFP and SIINFEKL (OVA257-264 peptide, SEQ ID NO: 15) to test MHC class I peptide presentation. Specifically, pCMV-Tag 2B.EGFP.SIINFEKL contains, in the following order, a CMV enhancer, a CMV promoter, a T3 promoter, MCS, ATG start codon, a FLAG-tag, EGFP, SIINFEKL (SEQ ID NO: 15), stop codons, a T7 promoter, a SV40 poly(A) signal, an f1 ori, an AmpR promoter, a NeoR/KanR, a HSV TK poly(A) signal, and ori sequences.

pCMVTag2B-RdRp-EGFP-SIINFEKL is a 7,821 base pair circular plasmid and contains EGFP, SIINKEKL (SEQ ID NO: 15), and SarS-COV-2 RdRp (non-structural protein subunit 12 (nsp12), codon-optimized). Specifically, the pCMVTag2B-RdRp-EGFP-SIINFEKL contains, in the following order, a human CMV enhancer, a CMV promoter, a T3 promoter, a Kozak consensus, SarS-CoV-2 RdRp, MCS, EGFP, SIINKEKL (SEQ ID NO: 15), stop codons, a T7 promoter, a SV40 poly(A) signal, an f1 ori, an AmpR promoter, an SV40 promoter, a NeoR/KanR, a HSV TK poly(A) signal, and ori sequences. The SarS-CoV-2 RdRp sequence contains Fbw7 degron, APCC1, APC/C 2 (D Box), APCC3 (D Box), and APCC4 (ABBAyCDC20). “D box” refers to sequence RxxL (SEQ ID NO: 16), wherein the two middle amino acids (i.e., the “x”s) can be any amino acid.

The modified RdRp proteins can be created using any suitable means. The plasmids described herein are merely exemplary plasmids used to determine RdRp expression via fluorescence and MHC class I expression. The recombinant vectors of an aspect of the invention can be co-administered with any vaccine. For example, the recombinant vectors of an aspect of the invention can be co-administered with a SARS-CoV-2 vaccine. The recombinant vectors of an aspect of the invention can be co-administered with an mRNA vaccine. The recombinant vectors of an aspect of the invention can be co-administered with BNT162b2 (COMIRNATY), JNJ-78436735, or mRNA-1273.

The vectors of an aspect of the invention can deliver the RdRp to the cells by any suitable means. For example, in an aspect of the invention, the RdRp can be delivered by transfection (e.g., mRNA transfection) or by introduction to the cells using a viral carrier.

Any suitable viral carrier can be used in an aspect of the invention, for example, an adenovirus, a poxvirus, a retrovirus, a herpes simplex virus, or a baculovirus. In an aspect of the invention, the viral carrier is an adenovirus.

Aspects, including embodiments, of the subject matter described herein may be beneficial alone or in combination, with one or more other aspects or embodiments. Without limiting the foregoing description, certain non-limiting aspects of the disclosure numbered 1-24 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A recombinant vector comprising one or more polynucleotides encoding at least one modified ribonucleic acid (RNA) dependent RNA polymerase (RdRp), wherein the modified RdRp comprises:
(a) at least one fragment of RdRp;
(b) RdRp and a regulatory protein that enhances proteasomal degradation, wherein the RdRp is conjugated or fused to the regulatory protein directly or indirectly via a linker;
(c) RdRp and a peptide sequence rich in proline (P), glutamic acid (E), serine (S), and threonine (T) (PEST sequence) that enhances proteasomal degradation; or
(d) a combination of two or more of (a)-(c).

(2) The vector of aspect 1, wherein the RdRp of (a), (b), or (c) is a corona virus RdRp.

(3) The vector of aspect 1, wherein the RdRp of (a), (b), or (c) is SARS-CoV-2 RdRp.

(4) The vector of any one of aspects 1-3, wherein the at least one fragment of RdRp comprises at least 100 amino acids.

(5) The vector of any one of aspects 1-4, wherein the at least one fragment of RdRp comprises no more than 950 amino acids.

(6) The vector of any one of aspects 1-5, wherein the at least one fragment of RdRp comprises at least one of SEQ ID NOs: 1-12.

(7) The vector of any one of aspects 1-6, wherein the vector comprises:
(a) SEQ ID NOs: 1, 8, and 9;
(b) SEQ ID NOs: 2 and 6;
(c) SEQ ID NOs: 4 and 7;
(d) SEQ ID NOs: 10 and 12;
(e) SEQ ID NOs: 1, 7, 8, 9, and 10; or
(f) a combination of two or more of (a)-(e).

(8) The vector of any one of aspects 1-3, wherein the regulatory protein is ubiquitin.

(9) The vector of aspect 1 or 8, wherein the regulatory protein is conjugated or fused to the N-terminus of RdRp.

(10) The vector of aspect 1 or 8, wherein the regulatory protein is conjugated or fused to the C-terminus of RdRp.

(11) The vector of any one of aspects 1-3 or 8-10, wherein the linker is an Alanine-Arginine linker.

(12) The vector of any one of aspects 1-3, wherein the PEST sequence comprises at least 10 amino acids and wherein at least 50% of the total amino acids in the PEST sequence comprise P, E, S, or T.

(13) The vector of any one of aspects 1-3, wherein the PEST sequence has at least 90% sequence identity to SEQ ID NO: 14.

(14) The vector of any one of aspects 1-3, 12, or 13, wherein the PEST sequence is conjugated or fused to the N-terminus of RdRp.

(15) The vector of any one of aspects 1-3, 12, or 13, wherein the PEST sequence is conjugated or fused to the C-terminus of RdRp.

(16) A pharmaceutical composition comprising the vector of any one of aspects 1-15 and a pharmaceutically acceptable carrier.

(17) The pharmaceutical composition of aspect 16, wherein the pharmaceutical composition is formulated for subcutaneous injection.

(18) A method of destabilizing SARS-CoV-2 RdRp in a cell, the method comprising contacting a cell with the vector according to any one of aspects 1-15.

(19) A method of increasing immune response to SARS-CoV-2 virus in a subject, the method comprising administering the vector according to any one of aspects 1-15 or the pharmaceutical composition of aspect 16 or 17 to the subject.

(20) A method of increasing cytotoxic T lymphocyte (CTL) response to SARS-CoV-2 virus in a subject, the method comprising administering the vector according to any one of aspects 1-15 or the pharmaceutical composition of aspect 16 or 17 to the subject.

(21) A method of increasing Major Histocompatibility Complex (MHC) class I immune response to SARS-CoV-2 virus in a subject, the method comprising administering the vector according to any one of aspects 1-15 or the pharmaceutical composition of aspect 16 or 17 to the subject.

(22) The method of any one of aspects 19-21, wherein the vector is administered subcutaneously.

(23) The method of any one of aspects 19-22, wherein the vector is administered more than once to the subject.

(24) The method of any one of aspects 19-23, wherein the subject is a human.

The following example further illustrates the invention but, of course, should not be construed as in any way limiting its scope.

### EXAMPLE 1

This example demonstrates the production and desirable immunity response of vectors of the present invention.

### Materials and Methods

### Plasmids

Constructs of the present invention were generated by PCR as described previously (Kwun, et al., J. Virol. 81:8225-8235 (2007)). FIGS. 1A and 1B show schematics of plasmids of aspects of the invention. As there are currently no standard ways of determining RdRp expression, EGFP was used to test fluorescence expression level and the chicken ovalabumin epitope SIINFEKL (OVA257-264 peptide, SEQ ID NO: 15) was used to test MHC class I peptide presentation. For SarS-CoV-2 RdRp, non-structural protein subunit 12 (nsp12) was used. The SarS-CoV-2 RdRp sequence contains Fbw7 degron, APCC1, APC/C 2 (D Box), APCC3 (D Box), and APCC4 (ABBAyCDC20). A destabilized EGFP was amplified by PCR using primers (pd1EGFP-sense, GGA TCC GCC ACC ATG GTG AGC AAG GGC GAG GAG CTG (SEQ ID NO: 19); pd1EGFP-antisense, GAA TTC CAC ATT GAT CCT AGC AGA AGC ACA (SEQ ID NO: 20)) and inserted into BamHI/EcoRI sites in the N-terminus of the constructs. SIINFEKL (SEQ ID NO: 15) was introduced into HindIII/XhoI sites using direct ligation of following primers: SIIN-Forward 5′-AAG CTT AGC ATA ATT AAT TTC GAA AAG CTC TAA GCG GCC GCG CTC GAG-3′ (SEQ ID NO: 21); SIIN-Reverse 5′-CTC GAG CGC GGC CGC TTA GAG CTT TTC GAA ATT AAT TAT GCT AAG CTT-3′ (SEQ ID NO: 22). Correct insert sequences were determined by DNA sequencing for all plasmids.

### Analysis of GFP Fluorescence (Protein Turnover)

HEK293 cells were grown in Dulbeccos' Modified Eagle Medium (DMEM) supplemented with 10% FBS and transfected with Lipofectamine-2000 (Invitrogen), with the constructs. To examine inhibition of GFP turnover, pd1EGFP-NI (Kwun, et al., (2007)) encoding a destabilized EGFP with an estimated half-life (t1/2) of 1 h was used for constructing fusions to CR subdomains or constructs at the N-terminus of EGFP. Samples were harvested and lysed in buffer (50 mM Tris-HCl [pH 8.0], 150 mM NaCl, 0.1% SDS, 3 mM EDTA, 1% Triton X-100, 1 mM NaF, and 1 mM Na orthovanadate) supplemented with proteinase inhibitors.

### Flow Cytometry Analysis for Antigen Presentation

HEK293 cells stably expressing the mouse class I allele H-2Kb were grown in DMEM with 10% FBS supplemented with 0.5 mg/ml G418 (HyClone Laboratories, Inc.). For flow cytometry analysis, 293KbC2 cells were transfected with 1 μg of the constructs and harvested and washed with PBS 24 hrs after transfection and stained with allophycocyanin (APC) or phycoerytherin (PE) anti-mouse MHC class I Kb-SIINFEKL (25-D1.16, SEQ ID NO: 15, eBioscience) for 1h at 4 degrees C. Cells were washed twice with PBS and analyzed using a BD LSRFORTESSA™ Cell Analyzer. For each experiment, gating was performed for positive EGFP fluorescence, and 30,000 events were collected and analyzed. All experiments were repeated at least three times for reproducibility, with representative experiments shown. Similar procedures were used for the B16 melanoma cells.

### Fluorescence Microscopy

Cells were analyzed using a Nikon TS100 with Spot insight digital camera or an Olympus AX70 epifluorescence microscope equipped with a Spot RT digital camera.

### Statistical Analysis

Data were compared by analysis of variance with paired student's t-test using Prism software (GraphPad). Values were considered significant at p<0.05.

### Discussion

RdRp was modified in three general ways: (1) fusion with ubiquitin at N-terminal; (2) fusion with a PEST sequence; and (3) fragmentation of RdRp. With these modifications, the increment of MHC class I immune response of RdRp (tested in vitro through SIINFEKL (SEQ ID NO: 15) peptide presentation) was determined. In summary, the modified RdRp resulted in destabilized RdRp which generated higher level of MHC class I peptide presentation and therefore will serve as antigens for vaccines, e.g., mRNA-based vaccines. The destabilized RdRp also is expected to increase the CTL response, which correlates to better vaccine protection. The data provide methods to modify RdRp to increase CTL immune response. The modified RdRp provides higher efficacy for vaccines, e.g., mRNA vaccines.

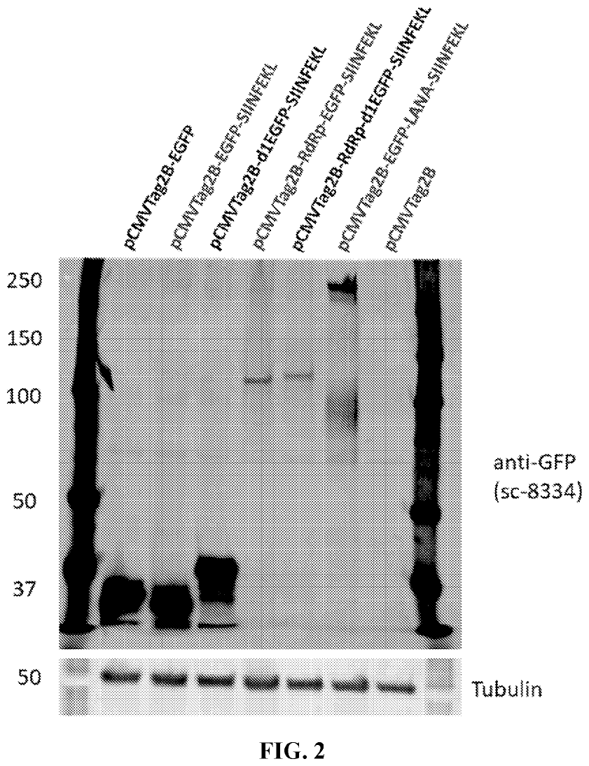

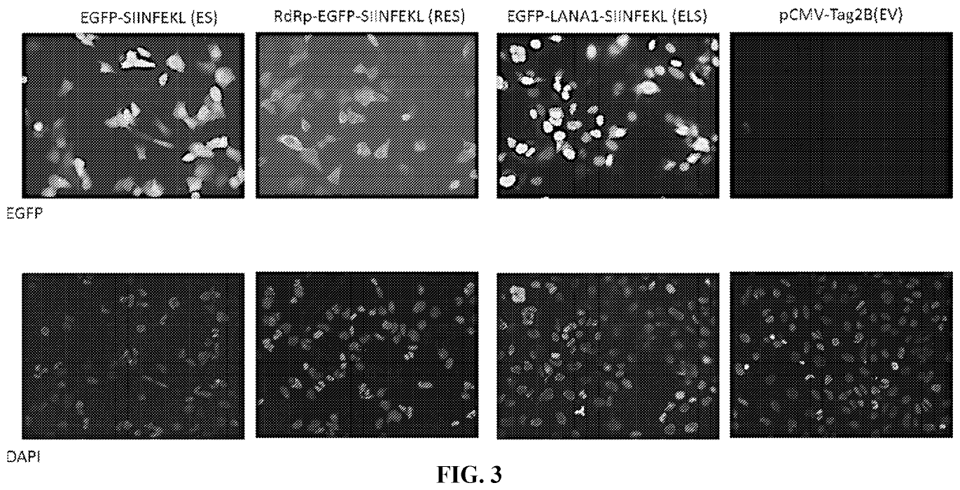

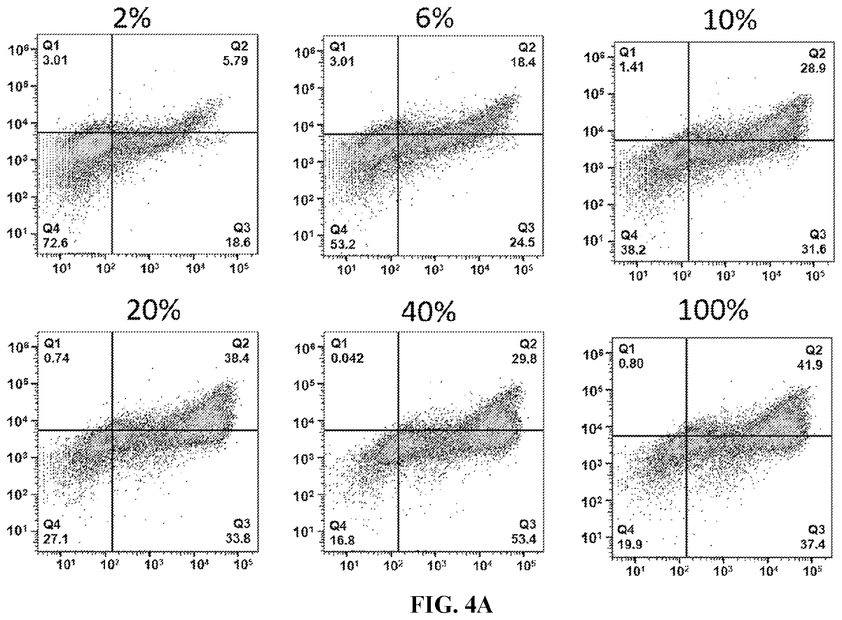

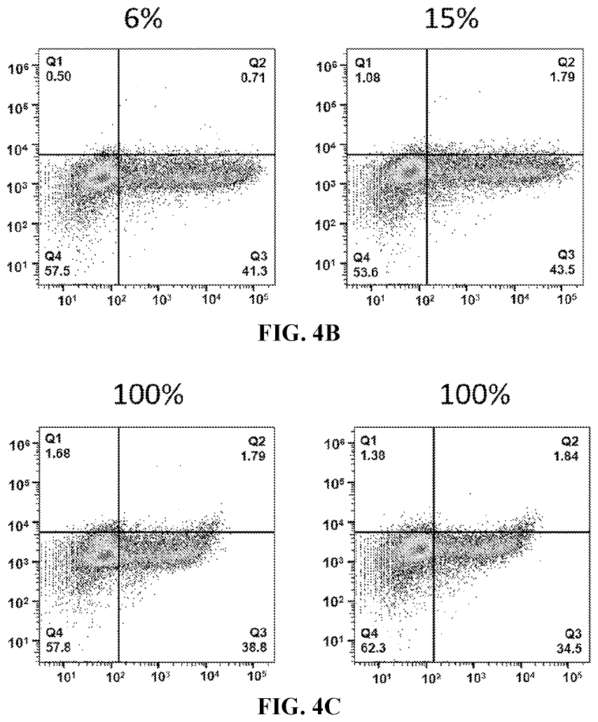

FIGS. 2 (Western Blot) and 3 (fluorescence images) are included as examples of the protein expression levels. FIGS. 4A-4C are sets of flow cytometry plots showing the EGFP levels versus SIINFEKL (SEQ ID NO: 15) presentation level of exemplary samples. At a similar GFP level, RdRp showed significantly lower MHC class I presentation. Without being bound to any particular theory, this could be due to the nature of virus to escape immune response. EGFP expression and SIINFEKL (SEQ ID NO: 15) presentation is saturated at around 20% for EGFP-SIINFEKL. Comparing FIGS. 4A and 4C, the GFP level is similar 156% EGFP-SIINFEKL vs. 100% RdRp-EGFP-SIINFEKL.

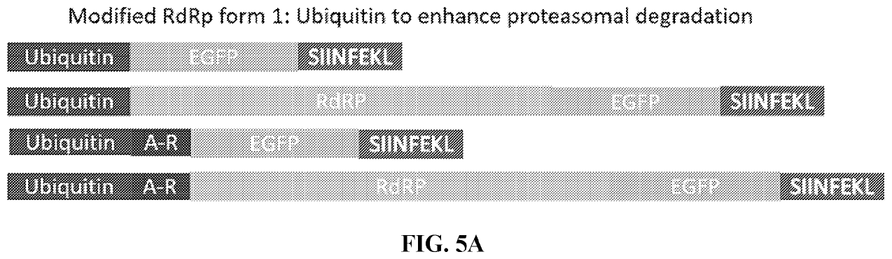

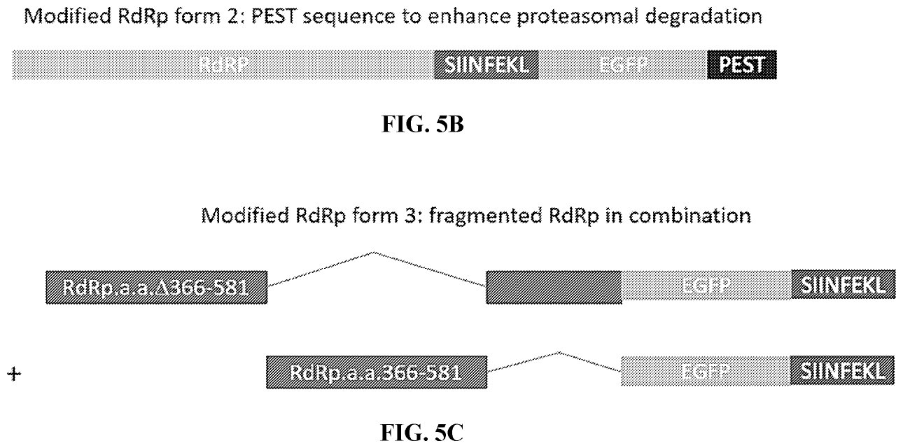

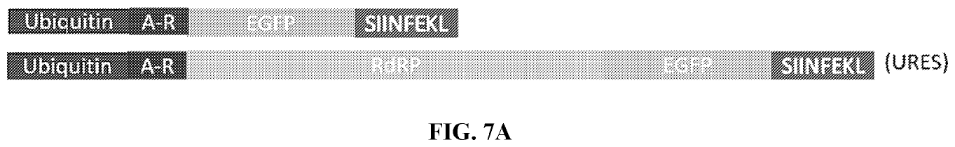

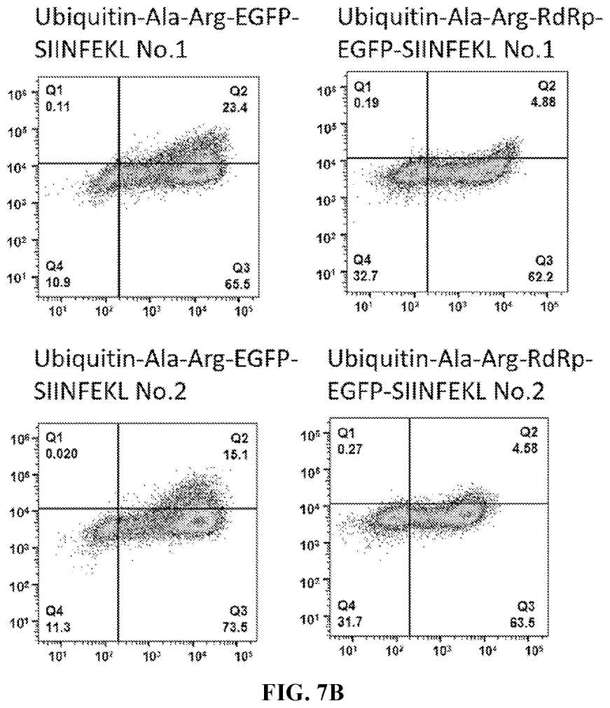

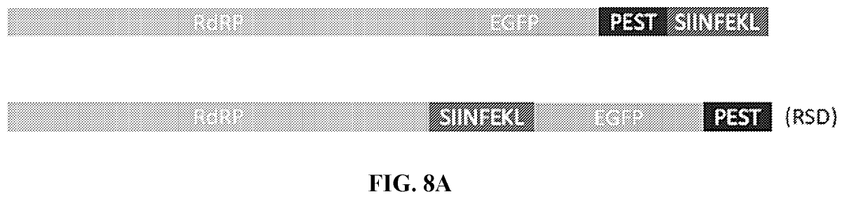

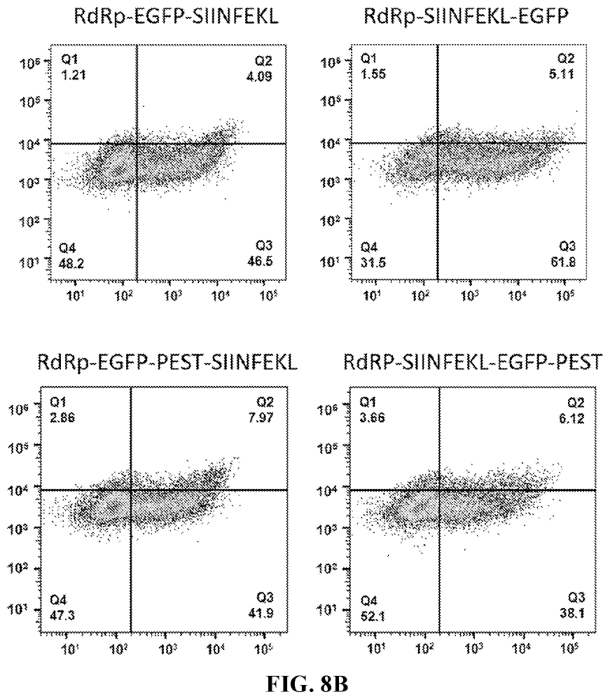

FIGS. 5A-5C, 6A, 7A, and 8A exemplary schematics showing aspects of the present invention.

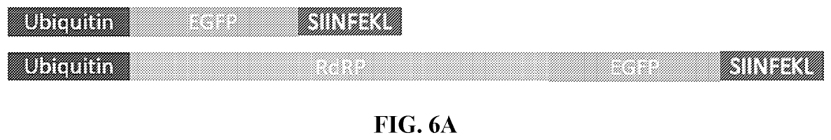

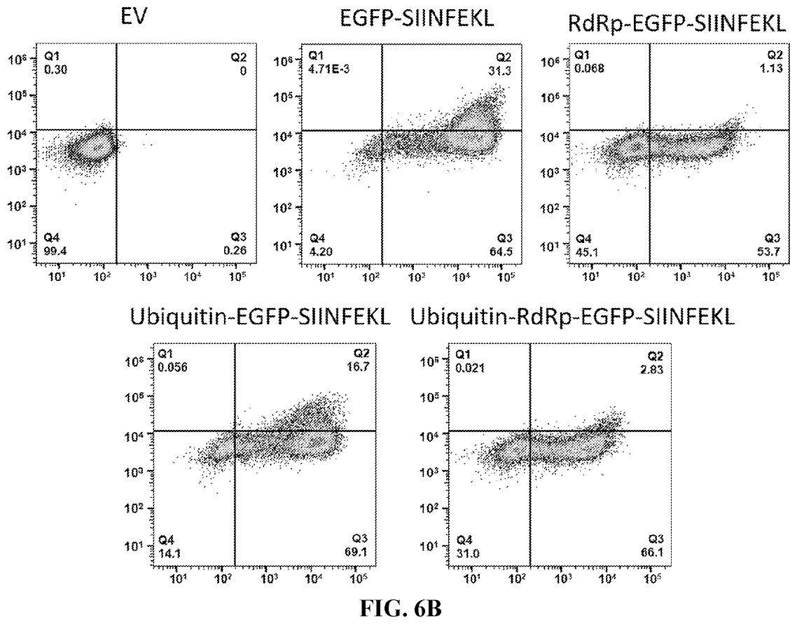

FIGS. 6B, 7B, and 8B are sets of flow cytometry plots showing MHC class I presentation of SIINFEKL (SEQ ID NO: 15).

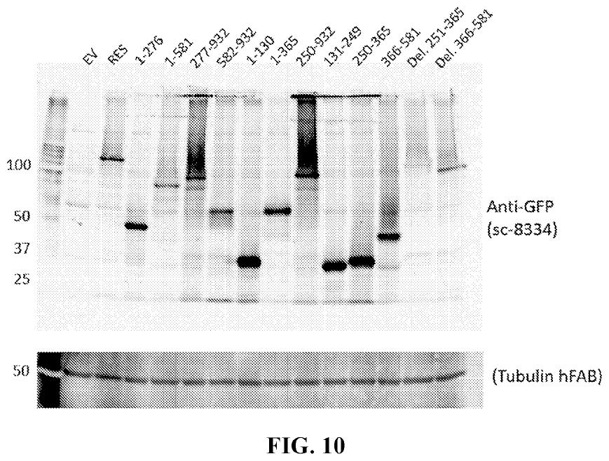

FIG. 10 is an exemplary gel following a Western blot analysis that screened for expression of RdRp fragment constructs.

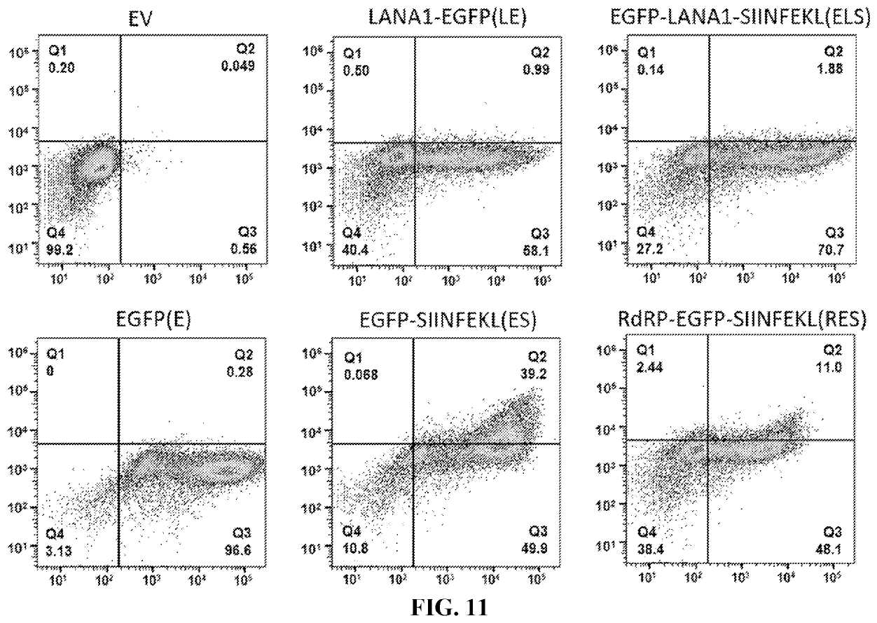

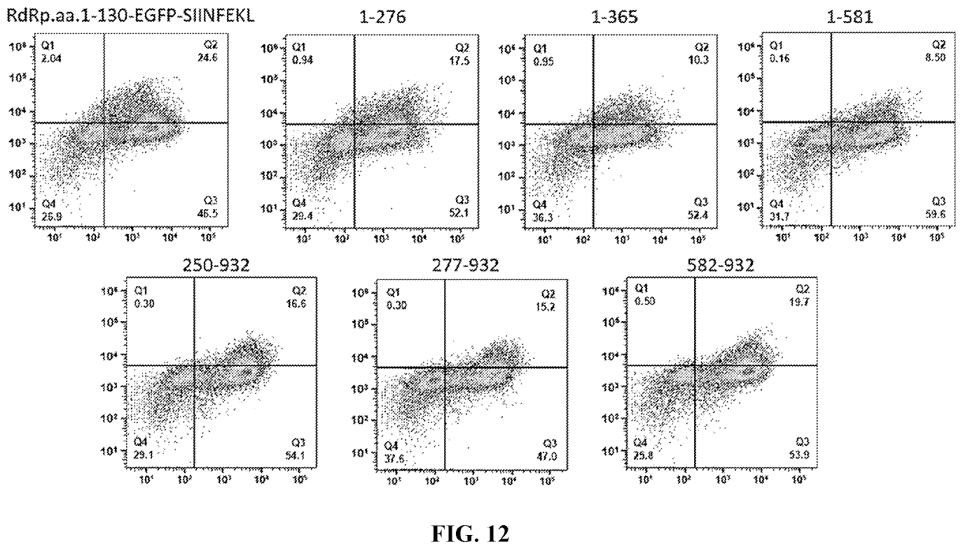

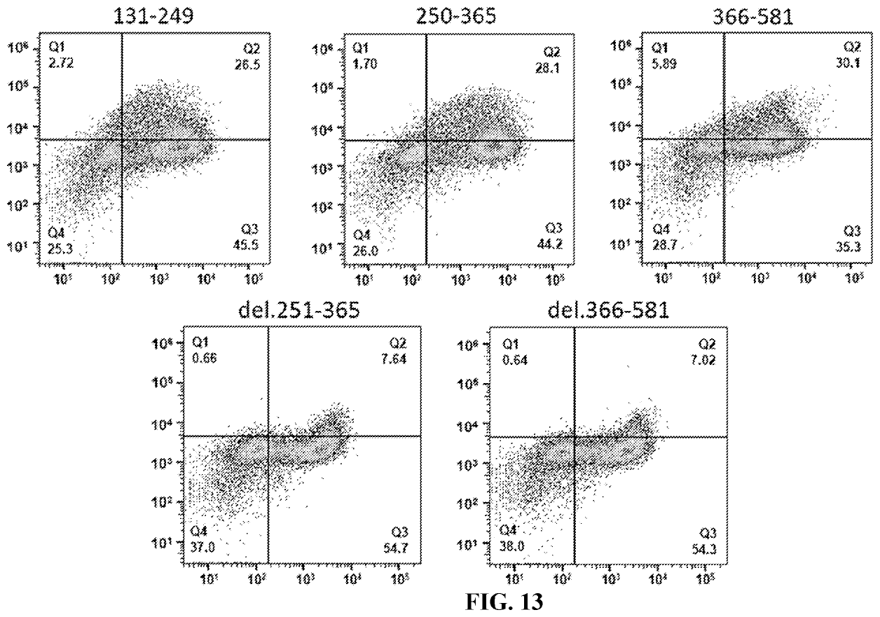

FIGS. 11-13 are sets of flow cytometry plots showing EGFP level and SIINFEKL (SEQ ID NO: 15) presentation of fragmented RdRp-EGFP-SIINFEKL constructs.

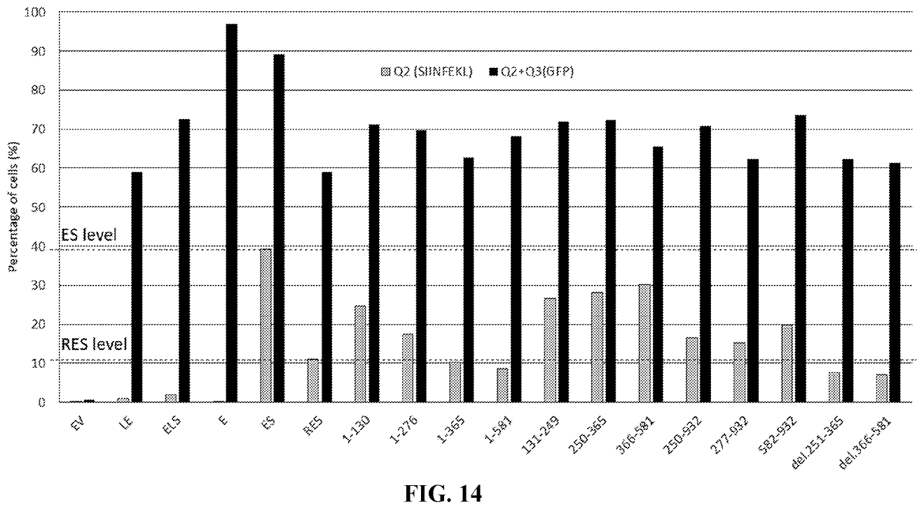

FIG. 14 is a graph comparing the EGFP level and SIINFEKL (SEQ ID NO: 15) presentation of fragmented RdRp-EGFP-SIINFEKL constructs to full length RdRp-EGFP-SIINFEKL.

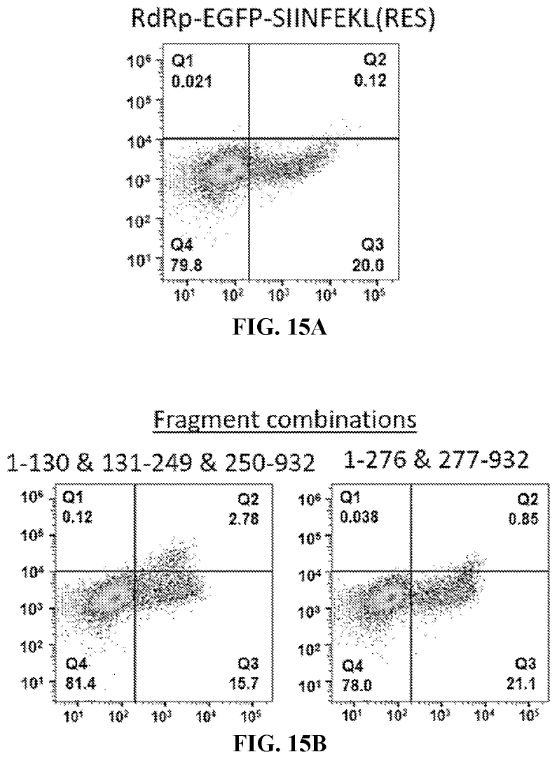

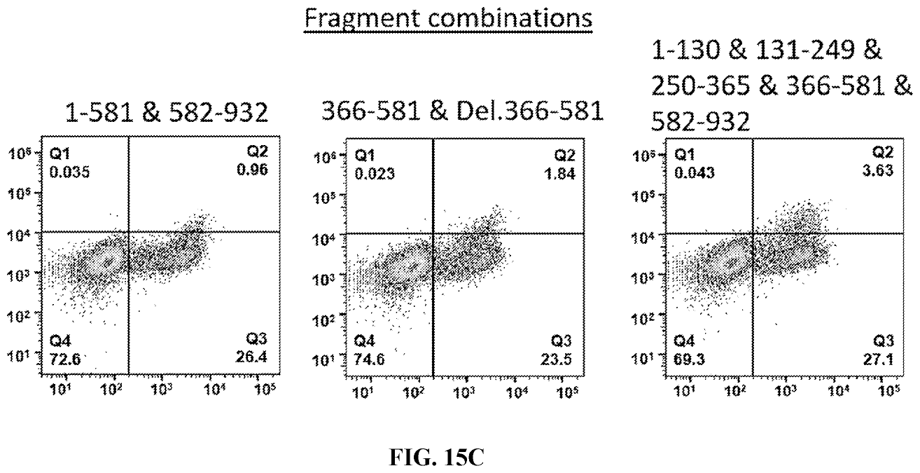

FIGS. 15A-15C are flow cytometry plots showing RdRp fragment combined (add up to a full-length RdRp) for SIINFEKL (SEQ ID NO: 15) presentation.

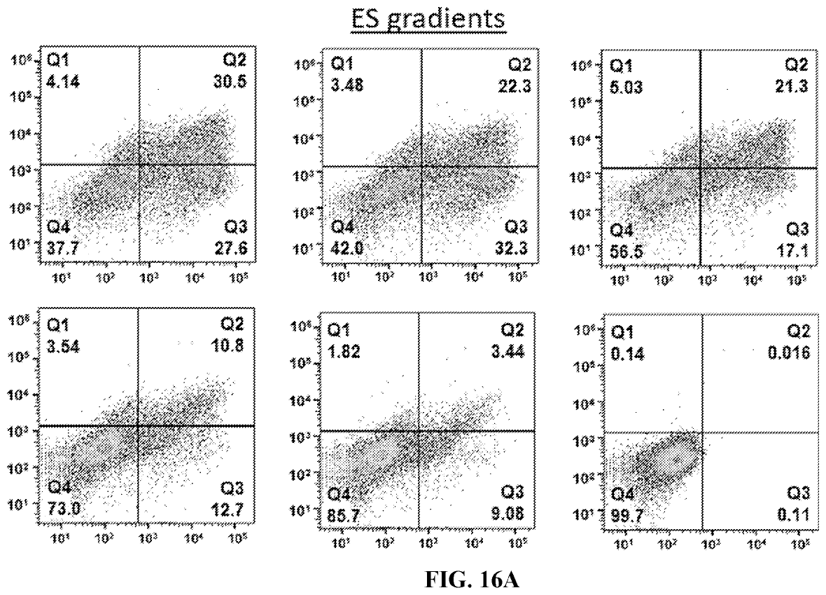

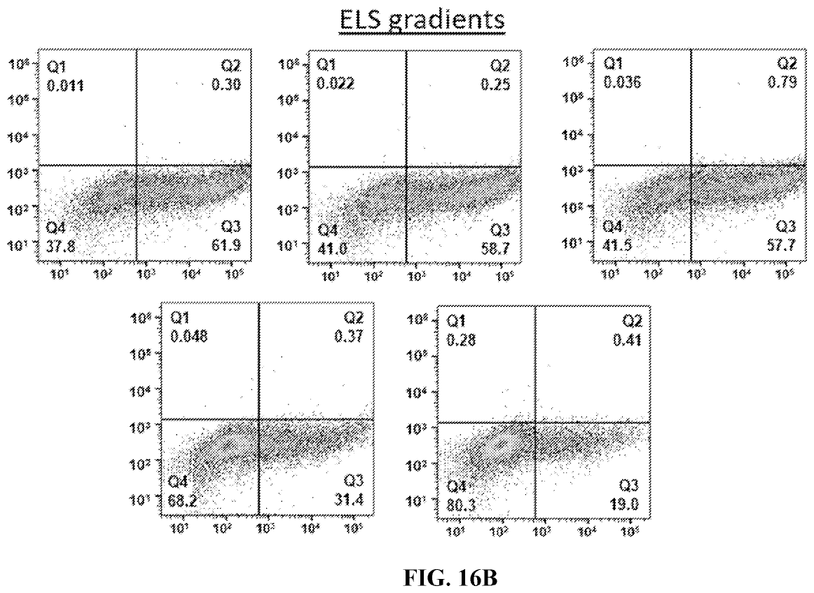

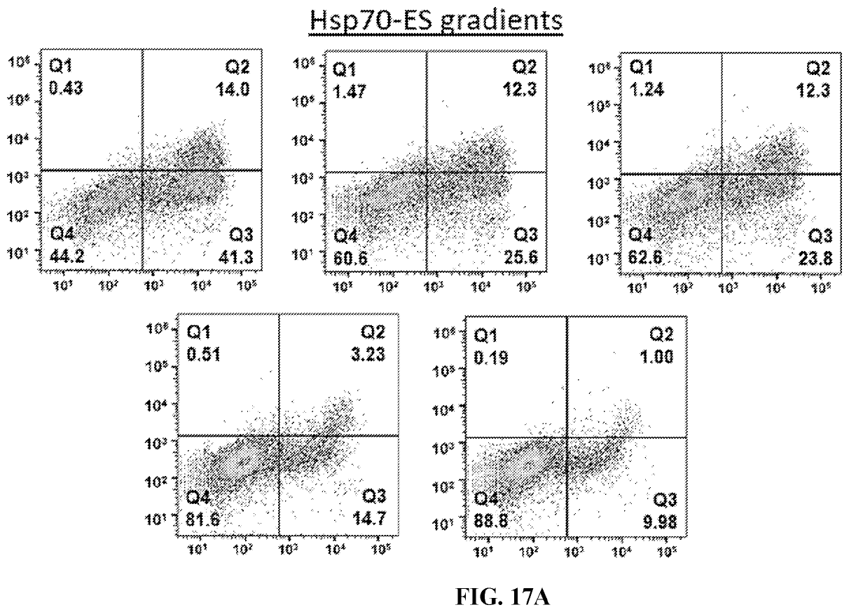

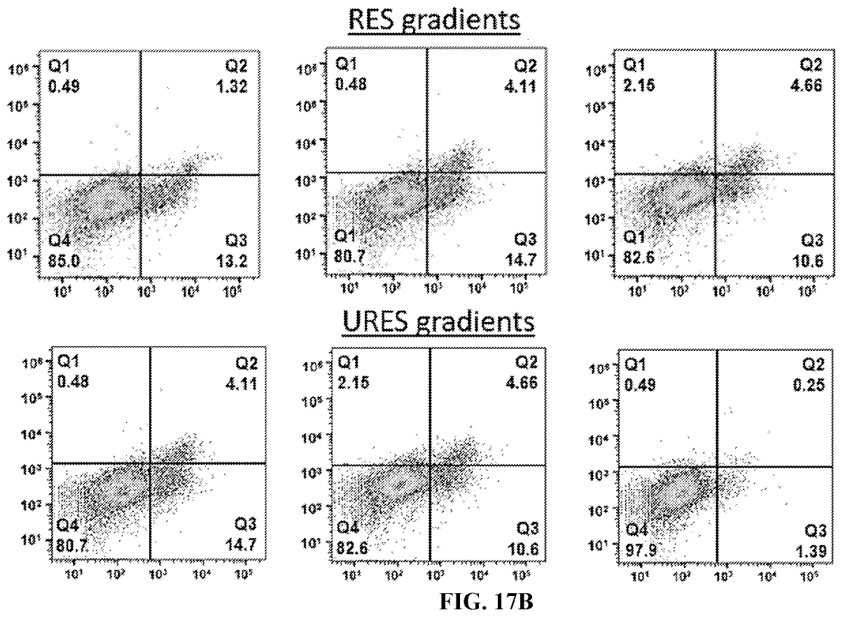

FIGS. 16A, 16B, 17A, and 17B are flow cytometry plots showing EGFP-SIINFEKL (SEQ ID NO: 15) and EGFP-LANAI as a control for RdRp-EGFP-SIINFEKL. Hsp70-EGFP-SIINFEKL has similar GFP level but lower SIINFEKL (SEQ ID NO: 15) presentation compared with RdRp-EGFP-SIINFEKL, which indicates that RdRp could actively suppress its peptide presentation.

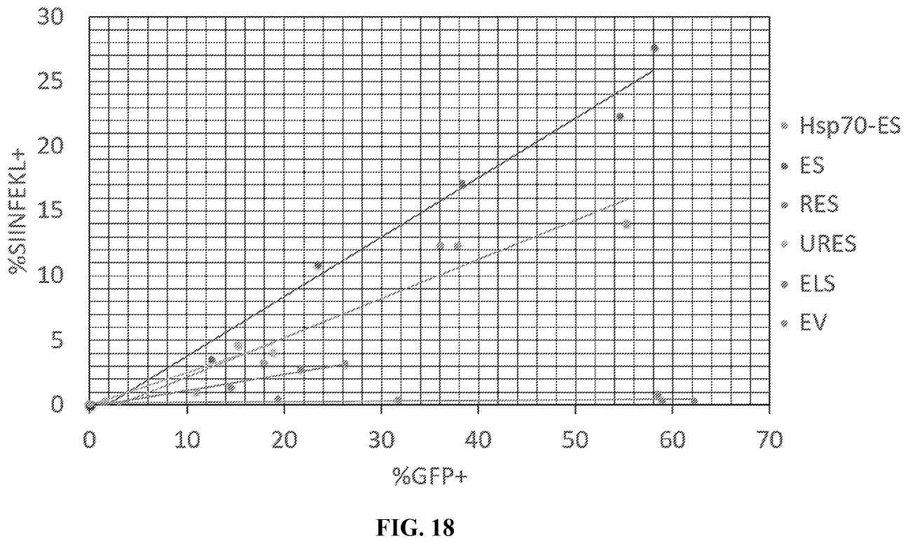

FIG. 18 is a graph showing GFP versus SIINFEKL (SEQ ID NO: 15) level and fitting lines for the shown constructs. The higher slope of the fitting line indicates higher SIINFEKL (SEQ ID NO: 15) presentation at the same GFP level (protein expression level). Ubiquitin-RdRp-EGFP-SIINFEKL shows higher SIINFEKL (SEQ ID NO: 15) presentation.

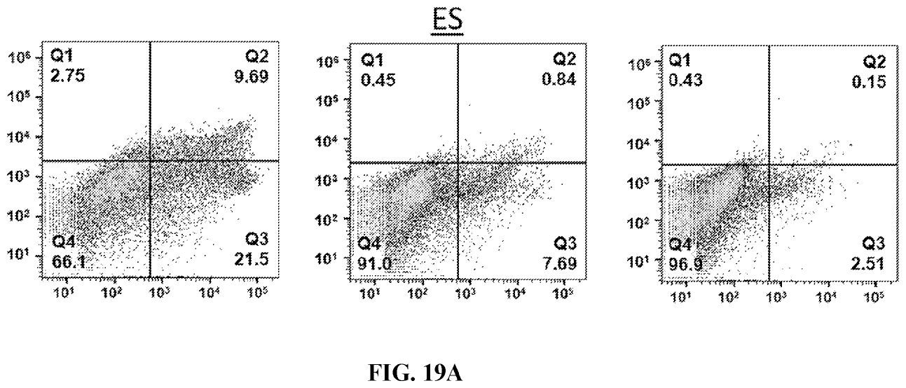

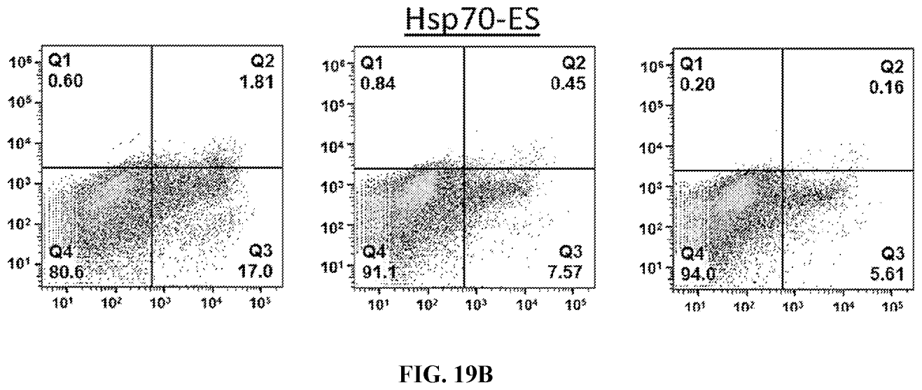

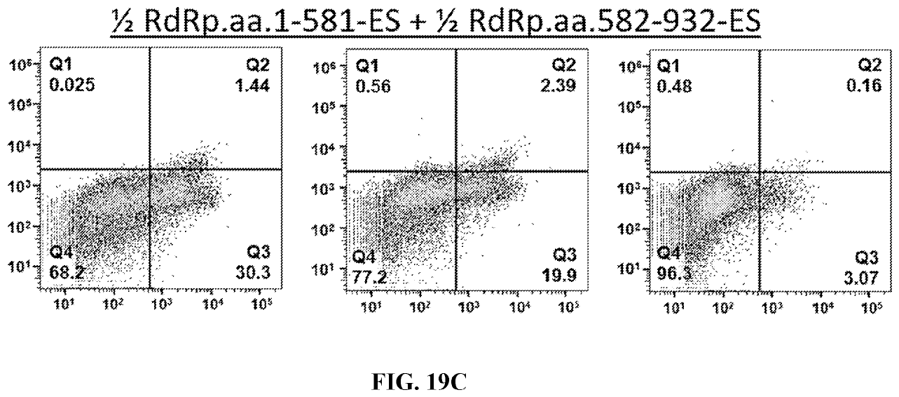

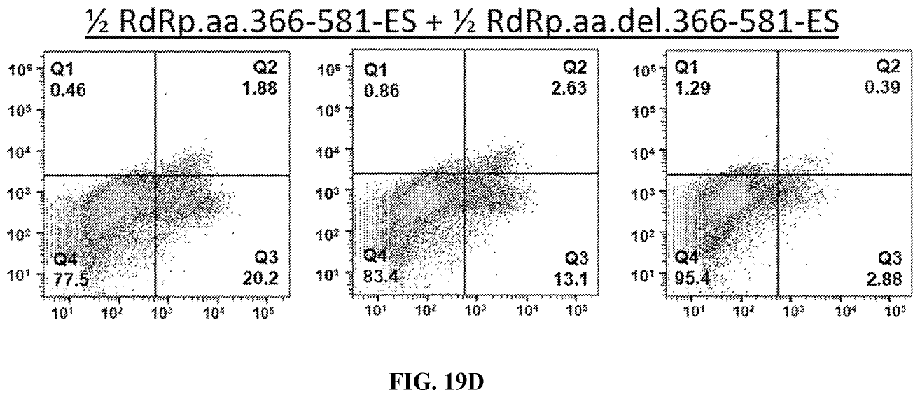

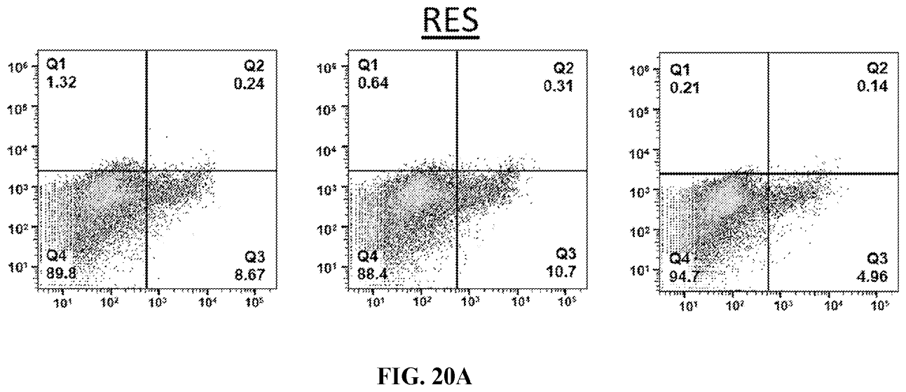

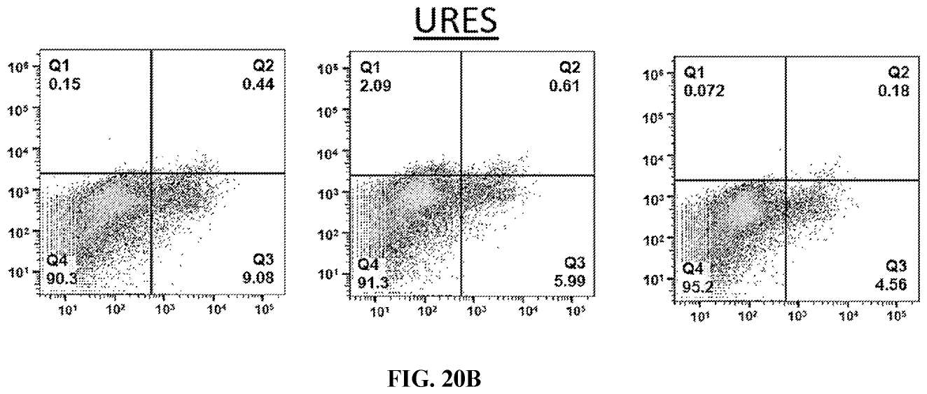

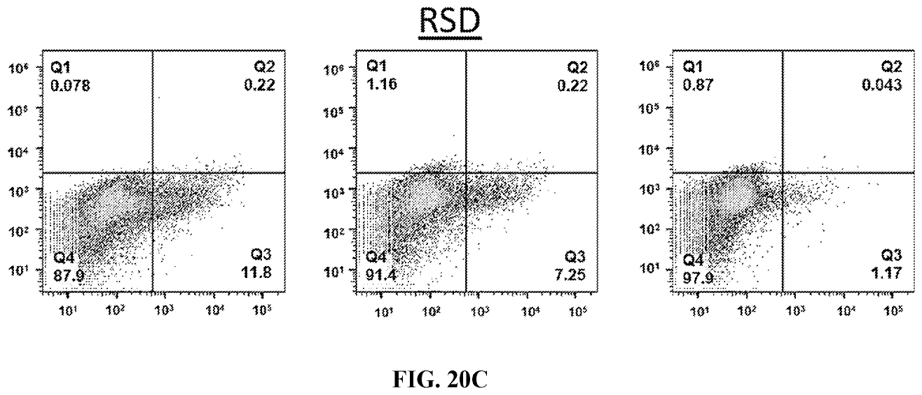

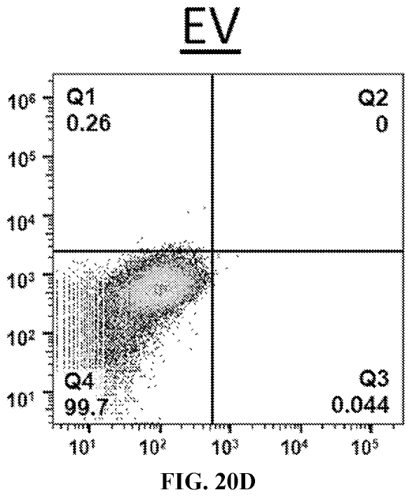

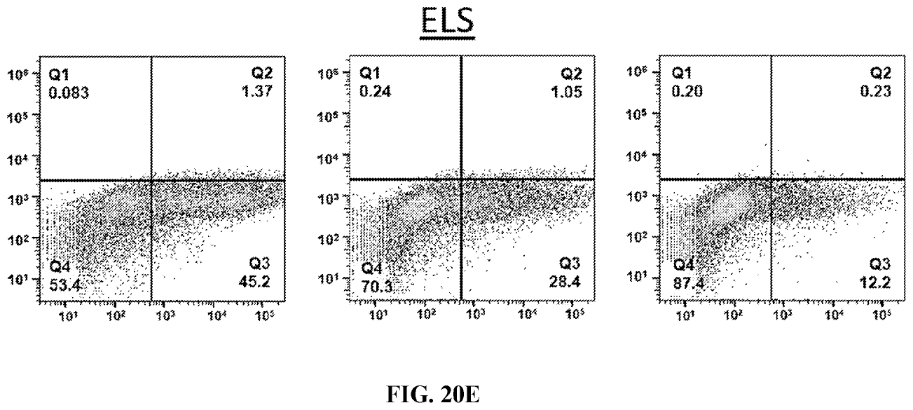

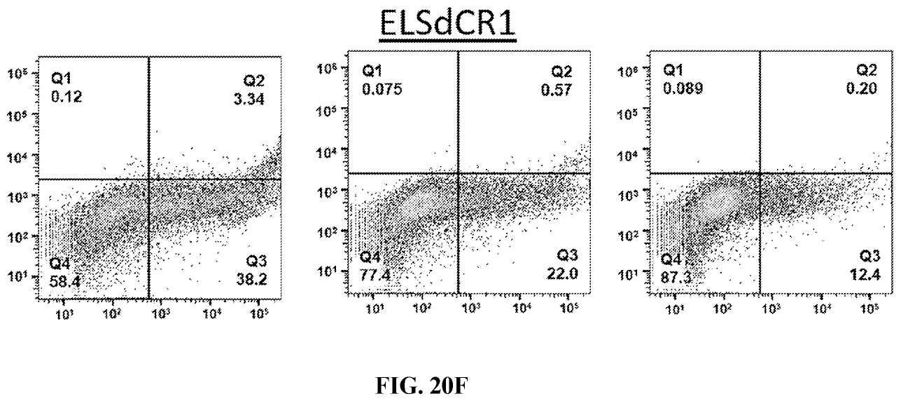

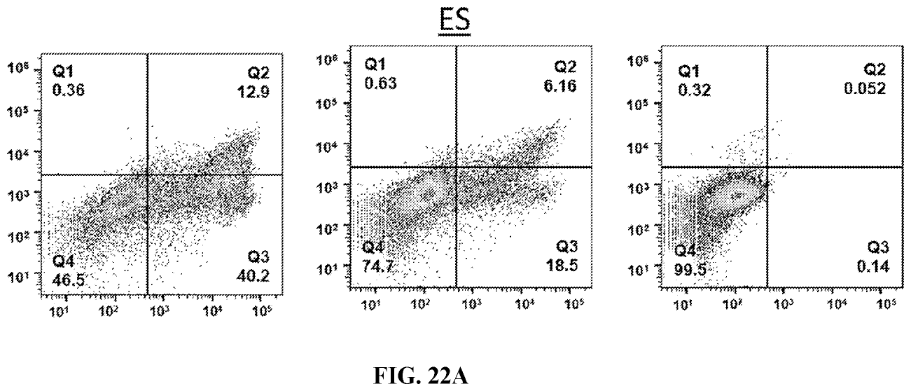

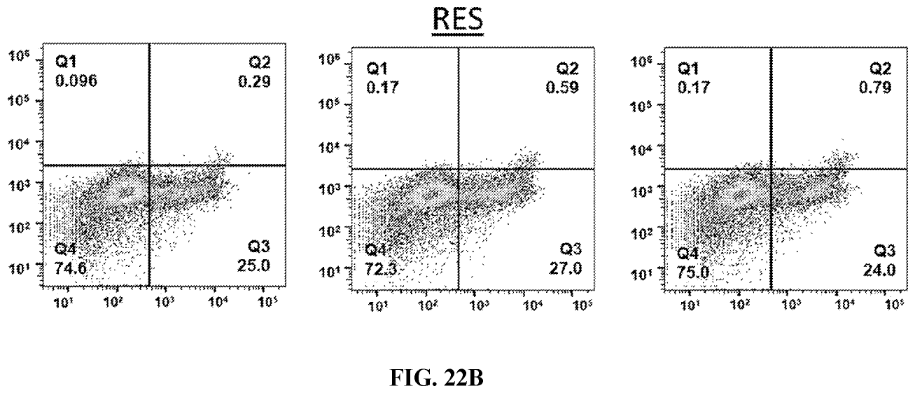

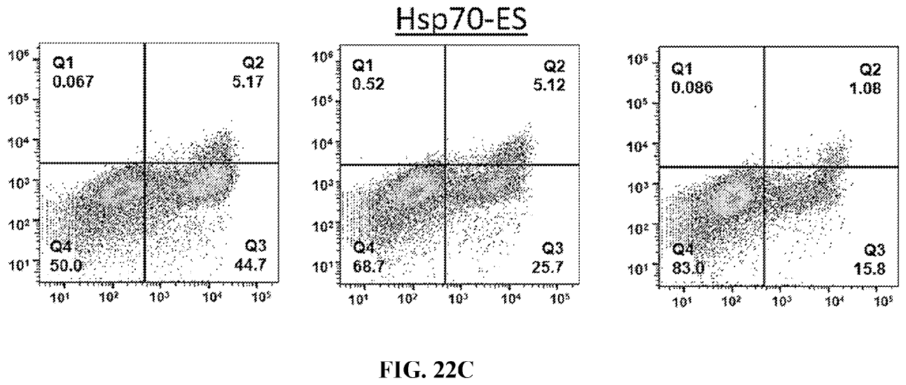

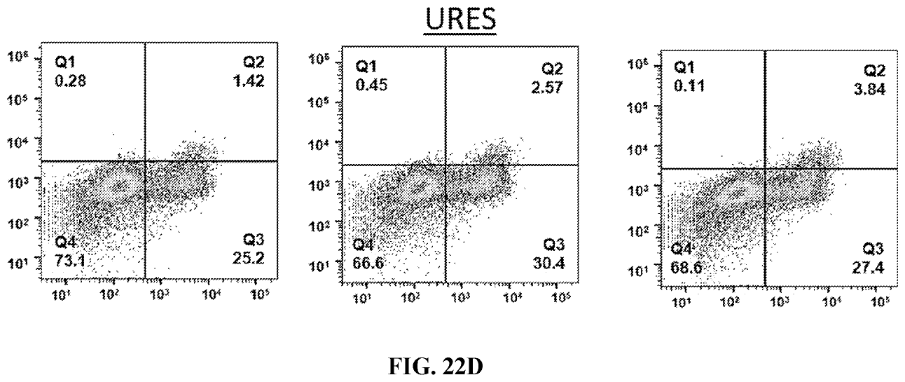

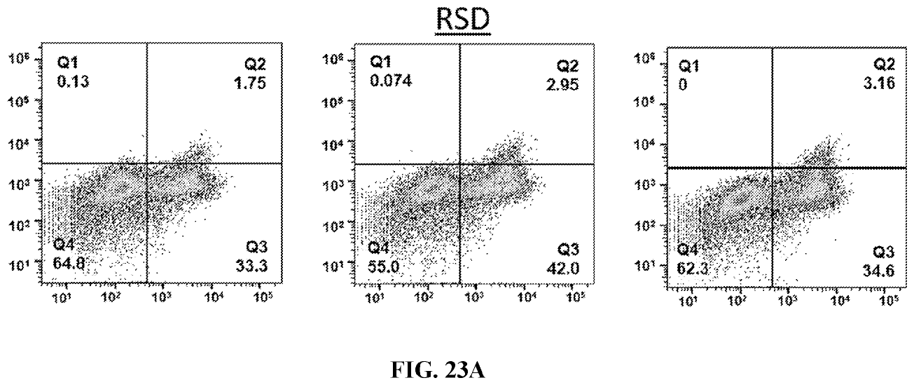

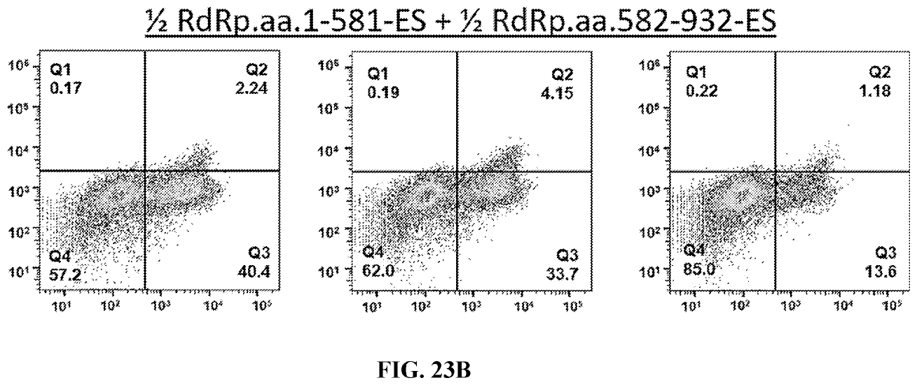

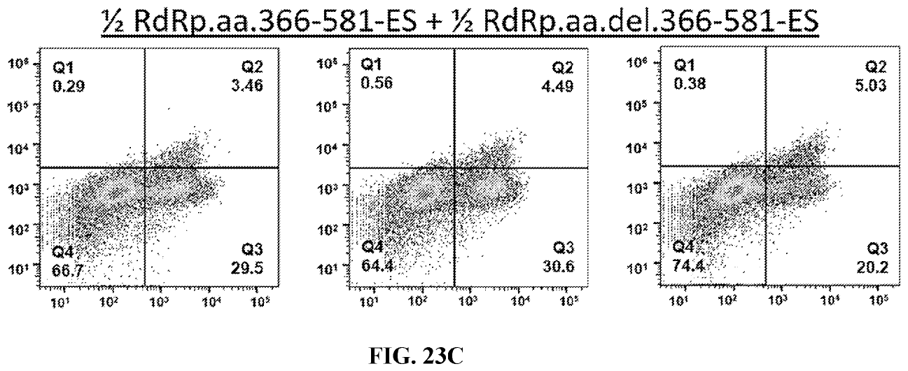

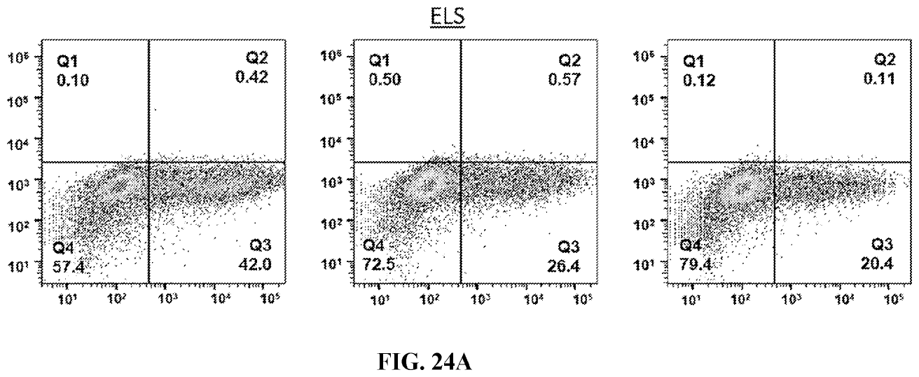

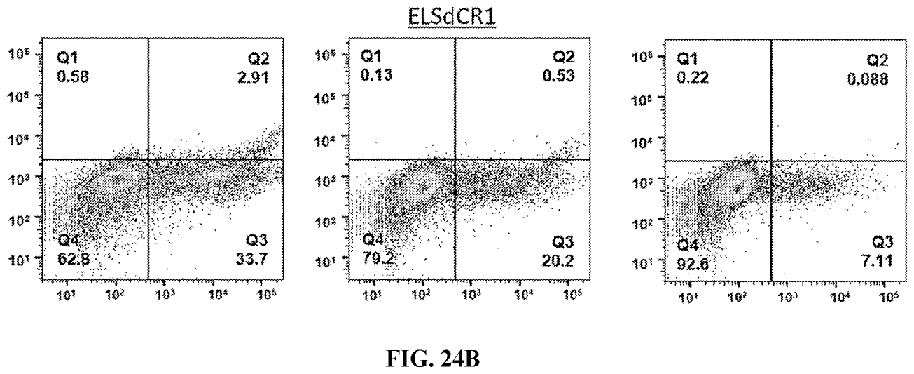

FIGS. 19A-19D, 20A-20F, 22A-22D, 23A-23C, and 24A-24B are flow cytometry plots showing for constructs at gradient (x3) concentrations. Combining FIGS. 19A-19D and FIGS. 20A-20F, fragmented RdRp combinations and ubiquitin increased SIINFEKL (SEQ ID NO: 15) presentation compared to full length RdRp.

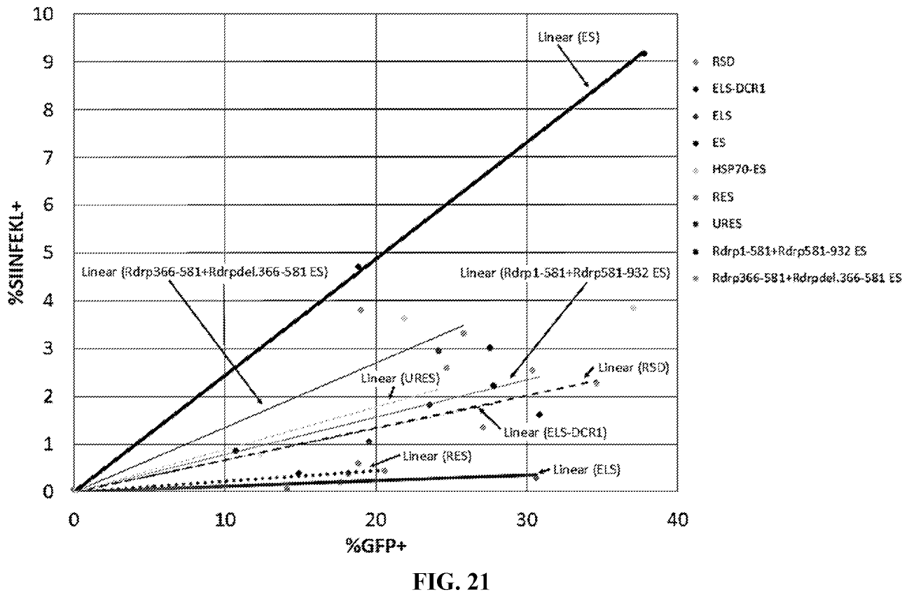

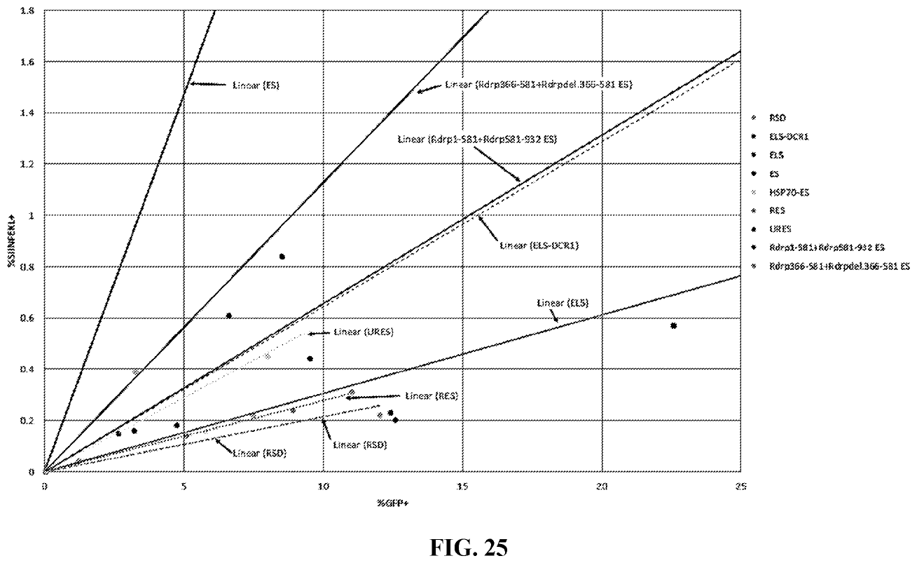

FIG. 21 is a graph showing GFP versus SIINFEKL (SEQ ID NO: 15) level and fitting lines for the shown constructs (1st). Higher slope of the fitting line indicates higher SIINFEKL (SEQ ID NO: 15) presentation at the same GFP level (protein expression level). This graph is similar to FIG. 18 but at a lower overall dose. Compared to RES (FL RdRp), all modified constructs except RSD presented higher SIINFEKL (SEQ ID NO: 15) peptides. Again, the SIINFEKL (SEQ ID NO: 15) presentation was increased in fragmented RdRp and Ubiquitin-RdRp constructs. FIG. 25 is a graph showing GFP versus SIINFEKL (SEQ ID NO: 15) level and fitting lines for the shown constructs (2nd).

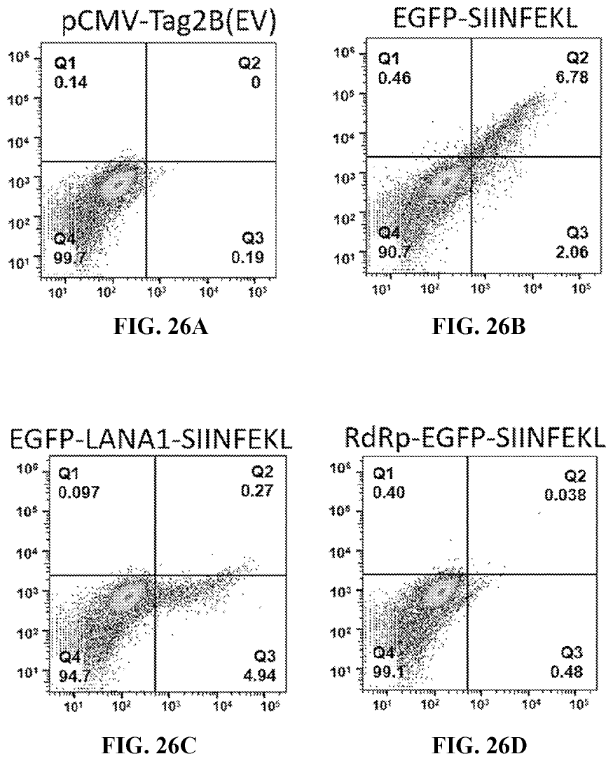

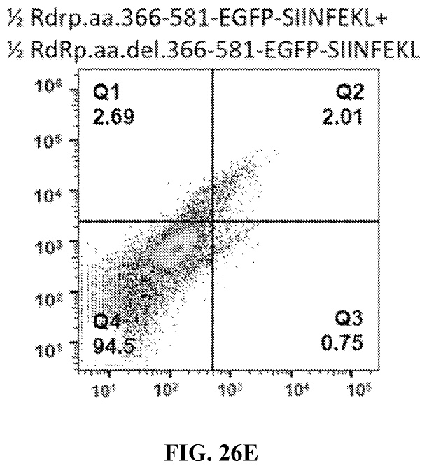

FIGS. 26A-26E are flow cytometry plots showing GFP versus SIINFEKL (SEQ ID NO: 15) level of constructs transfected into B16 melanoma cells (human). Fragmented RdRp showed higher SIINFKEL (SEQ ID NO: 15) presentation, in agreement with 293H2Kb cells. Similar to 293 cells, fragmented RdRp combination constructs showed higher SIINFEKL (SEQ ID NO: 15) presentation compared to full length RdRp, indicating a cell type independent effect.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments and aspects of this invention are described herein. Variations of those preferred embodiments and aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A recombinant vector comprising one or more polynucleotides encoding at least one modified ribonucleic acid (RNA) dependent RNA polymerase (RdRp), wherein the modified RdRp comprises:
(a) at least one fragment of RdRp;
(b) RdRp and a regulatory protein that enhances proteasomal degradation, wherein the RdRp is conjugated or fused to the regulatory protein directly or indirectly via a linker;
(c) RdRp and a peptide sequence rich in proline (P), glutamic acid (E), serine (S), and threonine (T) (PEST sequence) that enhances proteasomal degradation; or
(d) a combination of two or more of (a)-(c).

2. The vector of claim 1, wherein the RdRp of (a), (b), or (c) is a corona virus RdRp.

3. The vector of claim 1, wherein the RdRp of (a), (b), or (c) is SARS-CoV-2 RdRp.

4. The vector of claim 1, wherein the at least one fragment of RdRp comprises at least 100 amino acids.

5. The vector of claim 1, wherein the at least one fragment of RdRp comprises no more than 950 amino acids.

6. The vector of claim 1, wherein the at least one fragment of RdRp comprises at least one of SEQ ID NOs: 1-12.

7. The vector of claim 1, wherein the vector comprises:
(a) SEQ ID NOs: 1, 8, and 9;
(b) SEQ ID NOs: 2 and 6;
(c) SEQ ID NOs: 4 and 7;
(d) SEQ ID NOs: 10 and 12;
(e) SEQ ID NOs: 1, 7, 8, 9, and 10; or
(f) a combination of two or more of (a)-(e).

8. The vector of claim 1, wherein the regulatory protein is ubiquitin.

9. The vector of claim 1, wherein the regulatory protein is conjugated or fused to the N-terminus of RdRp.

10. The vector of claim 1, wherein the regulatory protein is conjugated or fused to the C-terminus of RdRp.

11. The vector of claim 1, wherein the linker is an Alanine-Arginine linker.

12. The vector of claim 1, wherein the PEST sequence comprises at least 10 amino acids and wherein at least 50% of the total amino acids in the PEST sequence comprise P, E, S, or T.

13. The vector of claim 1, wherein the PEST sequence has at least 90% sequence identity to SEQ ID NO: 14.

14. The vector of claim 1, wherein the PEST sequence is conjugated or fused to the N-terminus of RdRp.

15. The vector of claim 1, wherein the PEST sequence is conjugated or fused to the C-terminus of RdRp.

16. A pharmaceutical composition comprising the vector of claim 1 and a pharmaceutically acceptable carrier.

17. The pharmaceutical composition of claim 16, wherein the pharmaceutical composition is formulated for subcutaneous injection.

18. A method of destabilizing SARS-CoV-2 RdRp in a cell, the method comprising contacting a cell with the vector according to claim 1.

19. A method of increasing immune response to SARS-CoV-2 virus in a subject, the method comprising administering the vector according to claim 1 to the subject.

20.-21. (canceled)

22. The method of claim 19, wherein the vector is administered subcutaneously.

23.-24. (canceled)
